(19) United States
(12) Patent Application Publication
(10) Pub. No.: US 2022/0228168 A1
(43) Pub. Date: Jul. 21, 2022

(54) AFFINITY-BASED MULTIPLEXING FOR LIVE-CELL MONITORING OF COMPLEX CELL POPULATIONS

(71) Applicants: THE BROAD INSTITUTE, INC., Cambridge, MA (US); MASSACHUSETTS INSTITUTE OF TECHNOLOGY, Cambridge, MA (US)

(72) Inventors: Jacob Borrajo, Cambridge, MA (US); Paul Blainey, Cambridge, MA (US); Mohamad Najia, Cambridge, MA (US); Hong Anh Anna Le, Cambridge, MA (US); FuNien Tsai, Cambridge, MA (US)

(73) Assignees: THE BROAD INSTITUTE, INC., Cambridge, MA (US); MASSACHUSETTS INSTITUTE OF TECHNOLOGY, Cambridge, MA (US)

(21) Appl. No.: 17/607,097

(22) PCT Filed: Apr. 28, 2020
(86) PCT No.: PCT/US20/30286

§ 371 Date: Oct. 28, 2021

Related U.S. Application Data
(60) Provisional application No. 62/840,373, filed on Apr. 29, 2019.

(51) Int. Cl. C12N 15/86; C07K 14/005; C12N 15/10; G01N 33/50

(52) U.S. Cl. CPC C12N 15/86; C07K 14/005; C12N 15/1072; G01N 33/5005; C12N 2750/14123; C12N 2740/13031; C12N 2740/16023; C12N 2740/16031; C12N 2740/16222; C12N 2740/13023

(57) ABSTRACT

Compositions and methods for inducing and isolating virus-like particles (VLPs), and for allowing real-time assessment of VLP-captured analytes obtained from targeted living mammalian cells, are provided.

### CROSS REFERENCE TO RELATED APPLICATION

This application claims the benefit of U.S. Provisional Application No. 62/840,373, filed Apr. 29, 2019, entitled “Affinity-Based Multiplexing for Live-Cell Monitoring of Complex Cell Populations,” the entire contents of which are incorporated herein by reference.

### STATEMENT AS TO FEDERALLY SPONSORED RESEARCH

This invention was made with government support under Grant No. 1DP2HL141005 awarded by the National Institutes of Health. The government has certain rights in the invention.

### FIELD OF THE DISCLOSURE

The present disclosure relates to a method for live-cell monitoring of complex cell populations. More particularly, the present disclosure relates to compositions and methods for isolating and analyzing virus-like particles (VLPs) having cell line specific affinity-tagged envelopes.

### BACKGROUND OF THE INVENTION

While dramatic throughput advances in sequencing technologies have rendered transcriptome profiling via deep sequencing of even small volumes of cellular samples nearly routine, such methods tend to rely upon cell lysis to obtain samples for sequencing. A need therefore exists for a process that can yield transcriptome information from living cells, thereby allowing for expression profile monitoring of the same population(s) of cells throughout a time course.

### BRIEF SUMMARY OF THE INVENTION

The current disclosure relates, at least in part, to the discovery of compositions and methods capable of achieving assessment of living mammalian cell analytes across a time course, via induction of targeted virus like particle (VLP) generation in living cells and isolation/analysis of such VLPs to identify VLP-captured mammalian cell analytes.

In one aspect, the instant disclosure provides a mammalian cell harboring a virus like particle (VLP) producing protein and an epitope-tagged viral surface protein.

Another aspect of the instant disclosure provides a mammalian cell harboring an epitope-tagged virus like particle (VLP) producing protein.

An additional aspect of the instant disclosure provides a mammalian cell harboring a virus like particle (VLP) producing protein and a viral surface protein of a virus that differs from the virus of the virus like particle (VLP) producing protein (thereby generating a pseudotyped VLP).

In certain embodiments, the VLP is a non-enveloped virus VLP. Optionally, the non-enveloped virus harbors an engineered affinity tag. Optionally, the non-enveloped virus VLP is of an Adenoviridae, a Papovaviridae, a Parvoviridae, and/or an Anelloviridae family virus

In one embodiment, the virus like particle (VLP) producing protein is a retroviral gag protein or a viral gag-like protein. Optionally, the viral gag protein is a murine leukemia virus (MLV) gag protein, a retrovirus matrix protein, a rhabdovirus matrix (M) protein (optionally VSVM protein), a filovirus viral core protein (optionally an Ebola VP40 viral protein), a Rift Valley Fever virus N protein (optionally RVFV N Protein having GenBank serial number NP049344), a coronavirus M, E and/or NP protein (optionally GenBank serial number NP040838 for NP protein, GenBank serial number NP 040835 for M protein, GenBank serial number CAC39303 for E protein of Avian Infections Bronchitis Virus and GenBank serial number NP828854 for E protein of the SARS virus), a bunyavirus N protein (optionally the bunyavirus N protein of GenBank serial number AAA47114), an influenza M1 protein, a paramyxovirus M protein, an arenavirus Z protein (optionally a Lassa Fever Virus Z protein), and/or an AAV gag-like protein (optionally AAV1 capsid, AAV2 capsid, AAV3 capsid, AAV4 capsid, AAV5 capsid, AAV6 capsid, AAV7 capsid, AAV8 capsid, AAV9 capsid, AAV10 capsid, AAV11 capsid, AAV12 capsid, and/or AAV13 capsid), and/or combinations thereof. Optionally, the epitope-tagged viral surface protein is a Vesicular Stomatitis Virus (VSV) glycoprotein (VSV-G) or a mutagenized form of VSV-G. Optionally, the mutagenized form of VSV-G prevents VSV-G-mediated cellular uptake.

In certain embodiments, the epitope-tagged viral surface protein is an epitope-tagged viral envelope protein. Optionally, the epitope-tagged viral envelope protein is an epitope-tagged form of any of the following: a Vesicular Stomatitis Virus (VSV) glycoprotein, a retrovirus glycoprotein (optionally a human immunodeficiency virus (HIV) envelope glycoprotein (optionally HIVSF162 envelope glycoprotein of GenBank serial number M65024)), a simian immunodeficiency virus (SIV) envelope glycoprotein (optionally SIVmac239 envelope glycoprotein of GenBank serial number M33262), a simian-human immunodeficiency virus (SHIV) envelope glycoprotein (optionally SHIV-89.6p envelope glycoprotein of GenBank serial number U89134), a feline immunodeficiency virus (FIV) envelope glycoprotein (optionally FIV envelope glycoprotein of GenBank serial number L00607), a feline leukemia virus (FLV) envelope glycoprotein (optionally the FLV envelope glycoprotein of GenBank serial number M12500), a bovine immunodeficiency virus (BIV) envelope glycoprotein (optionally the BIV envelope glycoprotein of GenBank serial number NC001413), a bovine leukemia virus (BLV) envelope glycoprotein (optionally of GenBank serial number AF399703), an equine infectious anemia virus envelope glycoprotein (optionally the equine infectious anemia virus envelope glycoprotein of GenBank serial number NC001450), a human T-cell leukemia virus envelope glycoprotein (optionally the human T-cell leukemia virus envelope glycoprotein of GenBank serial number AF0033817), a mouse mammary tumor virus envelope glycoprotein (MMTV), a bunyavirus glycoprotein (optionally a Rift Valley Fever virus (RVFV) glycoprotein (optionally the RVFV envelope glycoprotein of GenBank serial number M11157)), an arenavirus glycoprotein (optionally a Lassa fever virus glycoprotein (optionally of GenBank serial number AF333969))), a filovirus glycoprotein (e.g., an Ebola virus glycoprotein (GenBank serial number NC002549)), a corona virus glycoprotein (optionally of GenBank serial number SARS coronavirus spike protein AAP13567), an influenza virus glycoprotein (optionally of GenBank serial number V01085), a paramyxovirus glycoprotein (optionally of GenBank serial number NC002728 for Nipah virus F and G proteins), a rhabdovirus glycoprotein (optionally of GenBank serial number NP049548)), an alphavirus glycoprotein (optionally of GenBank serial number AAA48370 for Venezuelan equine encephalomyelitis (VEE)), a flavivirus glycoprotein (optionally of GenBank serial number NC001563 for West Nile virus and/or a Hepatitis C Virus glycoprotein), and/or a Herpes Virus glycoprotein (optionally a cytomegalovirus glycoprotein), and/or combinations thereof.

In one embodiment, the epitope-tagged viral surface protein is Coronavirus gpE1, Coronavirus Peplomer Protein E1, Coronavirus Peplomer Protein E2 JHM, Hepatitis Virus (MHV), Glycoprotein E2, LaCrosse Virus Envelope Glycoprotein G1, Simian Sarcoma Virus Glycoprotein 70, Viral Envelope Glycoprotein gp55 (Friend Virus), and/or Viral Envelope Glycoprotein gPr90 (Murine Leukemia Virus).

In some embodiments, the epitope tag of the epitope-tagged viral surface protein is one or more of the following: FLAG (DYKDDDDK; SEQ ID NO: 3), 6×His (HHHHHH; SEQ ID NO: 4), HA (YPYDVPDYA; SEQ ID NO: 5), c-myc (EQKLISEEDL; SEQ ID NO: 6), V5 tag (GKPIPNPLLGLDST; SEQ ID NO: 7), AU1 tag (DTYRYI; SEQ ID NO: 8), AU5 tag (TDFYLK; SEQ ID NO: 9), Glu-Glu tag (EYMPME; SEQ ID NO: 10), OLLAS (SGFANELGPRLMGK; SEQ ID NO: 11), T7 tag (MASMTGGQQMG; SEQ ID NO: 12), VSV-G tag (YTDIEMNRLGK; SEQ ID NO: 13), E-Tag (GAPVPYPDPLEPR; SEQ ID NO: 14), S-Tag (KETAAAKFERQHMDS; SEQ ID NO: 15), HSV tag (SQPELAPEDPED; SEQ ID NO: 16), KT3 tag (KPPTPPPEPET; SEQ ID NO: 17), TK15 tag, GST tag, Protein A tag, CD tag, Strep-Tag (WSHPQFEK; SEQ ID NO: 18), MBP tag, CBD tag, Avi tag (CGLNDIFEAQKIEWHE; SEQ ID NO: 19), CBP tag, TAP tag, and/or SF-TAP tag.

In one embodiment, the mammalian cell is infected by a virus. Optionally, the mammalian cell is infected by AAV (and optionally adenovirus, HPV or other virus), or by a retrovirus. Optionally, the retrovirus is a lentivirus.

In some embodiments, the mammalian cell is a cell in culture

In one embodiment, the mammalian cell is a neuronal cell. Optionally, the mammalian cell is a primary cortical neuron. Optionally, the primary cortical neuron is an excitatory neuron or an inhibitory neuron.

In certain embodiments, the mammalian cell is a cell in vivo.

In some embodiments, the VLP producing protein and/or the epitope-tagged viral surface protein are produced by the mammalian cell via a genomically integrated nucleic acid sequence that encodes for the VLP producing protein and/or the epitope-tagged viral surface protein. Optionally, the nucleic acid sequence that encodes for the VLP producing protein and/or the epitope-tagged viral surface protein is under the control of a mammalian promoter. Optionally, the promoter is a CMV promoter, a SV40 promoter and/or a tissue-specific mammalian promoter. Optionally, the tissue-specific mammalian promoter is a mDIx, CamKII, Syn1, NSE, PDGF and/or Ta1 promoter. Optionally, the tissue-specific mammalian promoter is a CamKII promoter and/or a mDIx promoter.

An additional aspect of the instant disclosure provides a method for obtaining an expression profile of a living cell, the method involving: (a) providing a living cell; (b) introducing a nucleic acid sequence encoding for a VLP producing protein to the living cell, where introduction of the nucleic acid sequence encoding for a VLP producing protein is sufficient to induce budding of VLPs from the living cell; (c) isolating VLPs produced by the living cell via binding of a VLP protein; and (d) performing RNA sequencing upon the isolated VLPs, thereby obtaining expression profile information for the isolated VLPs, where the expression profile information for the isolated VLPs reflects the expression profile of the living cell, thereby obtaining an expression profile of the living cell.

In certain embodiments, the VLP protein of step (c) is the VLP producing protein. Optionally, the VLP producing protein is tagged. Optionally, the tag is an epitope tag.

In some embodiments, the VLP protein of step (c) is a capsid protein of the VLP or an envelope protein of the VLP.

In another embodiment, the VLP protein of step (c) is a host cell membrane protein. Optionally, the VLP protein of step (c) is an affinity-tagged host cell membrane protein.

In an additional embodiment, the capsid protein of the VLP or the envelope protein of the VLP is tagged. Optionally, the tag is an epitope tag.

In some embodiments, an antibody is used to bind the VLP protein in step (c).

In another embodiment, the antibody binds the VLP producing protein, a capsid protein of the VLP, and/or an envelope protein of the VLP.

Another aspect of the instant disclosure provides a method for obtaining an expression profile of a living cell, the method involving: (a) providing a living cell; (b) introducing a first nucleic acid sequence harboring a nucleic acid sequence encoding for a VLP producing protein and a second nucleic acid harboring a nucleic acid sequence encoding for an epitope-tagged viral surface protein to the living cell, where introduction of the first nucleic acid sequence harboring the nucleic acid sequence encoding for a VLP producing protein is sufficient to induce budding of VLPs from the living cell; (c) isolating VLPs produced by the living cell via binding of the epitope-tagged viral surface protein; and (d) performing RNA sequencing upon the isolated VLPs, thereby obtaining expression profile information for the isolated VLPs, where the expression profile information for the isolated VLPs reflects the expression profile of the living cell, thereby obtaining an expression profile of the living cell.

In certain embodiments, the living cell is a mammalian cell. Optionally, the living cell is a mammalian cell in culture.

In some embodiments, the living cell is a neuronal cell. Optionally, the neuronal cell is a primary cortical neuron. Optionally, the primary cortical neuron is an excitatory neuron or an inhibitory neuron.

In one embodiment, the living cell is a cell in vivo. Optionally, the living cell is a cell in a mouse model of disease. Optionally, the living cell is a cell in an engineered patient-derived xenograft (PDX) model for glioblastoma multiforme (GBM).

In some embodiments, the living cell is a living cell in a rat. Optionally, the living cell is a primary rat cortical neuron or a primary rat hippocampal neuron. Optionally, the living cell is obtained from microsurgically dissected tissue. Optionally the living cell is obtained from an E18 Sprague Dawley rat.

In some embodiments, the first nucleic acid sequence and the second nucleic acid sequence are present on the same nucleic acid construct.

In one embodiment, the first nucleic acid sequence and the second nucleic acid sequence are present on different nucleic acid constructs.

In certain embodiments, the first nucleic acid sequence and/or the second nucleic acid sequence are integrated in the living cell genome.

In one embodiment, the VLP producing protein and/or the epitope-tagged viral surface protein is under the control of a mammalian promoter. Optionally, the mammalian promoter is a CMV promoter, a SV40 promoter and/or a tissue-specific mammalian promoter. Optionally, the tissue-specific mammalian promoter is a CamKII promoter and/or a mDIx promoter.

An additional aspect of the instant disclosure provides a method for obtaining a first analyte profile for a first population of living cells and a second analyte profile for a second population of living cells, the method involving: (a) providing a first population of living cells; (b) introducing a first nucleic acid sequence harboring a nucleic acid sequence encoding for a VLP producing protein and a second nucleic acid harboring a nucleic acid sequence encoding for a first epitope-tagged viral surface protein to the first population of living cells, where introduction of the first nucleic acid sequence encoding for a VLP producing protein is sufficient to induce budding of VLPs from the first population of living cells; (c) providing a second population of living cells; (d) introducing the first nucleic acid sequence harboring a nucleic acid sequence encoding for a VLP producing protein and a second nucleic acid harboring a nucleic acid sequence encoding for a second epitope-tagged viral surface protein to the second population of living cells, where introduction of the first nucleic acid sequence comprising a nucleic acid sequence encoding for a VLP producing protein is sufficient to induce budding of VLPs from the second population of living cells; (e) isolating VLPs produced by the first population of living cells via binding of the first epitope-tagged viral surface protein; (f) obtaining a first analyte profile from the isolated VLPs of the first population of living cells; (g) isolating VLPs produced by the second population of living cells via binding of the second epitope-tagged viral surface protein; and (h) obtaining a second analyte profile from the isolated VLPs of the second population of living cells, thereby obtaining a first analyte profile for a first population of living cells and a second analyte profile for a second population of living cells.

In one embodiment, the analyte profile includes transcript information (e.g., transcriptome expression levels).

An additional aspect of the instant disclosure provides a method for obtaining a first analyte profile for a first population of living cells and a second analyte profile for a second population of living cells, the method involving: (a) providing a first population of living cells; (b) introducing a first nucleic acid sequence encoding for a VLP producing protein to the first population of living cells, where introduction of the first nucleic acid sequence encoding for a VLP producing protein is sufficient to induce budding of VLPs from the first population of living cells; (c) providing a second population of living cells; (d) introducing a second nucleic acid sequence encoding for a VLP producing protein to the second population of living cells, where introduction of the second nucleic acid sequence encoding for a VLP producing protein is sufficient to induce budding of VLPs from the second population of living cells; (e) isolating VLPs produced by the first population of living cells via binding of a first VLP protein; (f) obtaining a first analyte profile from the isolated VLPs of the first population of living cells; (g) isolating VLPs produced by the second population of living cells via binding of a second VLP protein; and (h) obtaining a second analyte profile from the isolated VLPs of the second population of living cells, thereby obtaining a first analyte profile for a first population of living cells and a second analyte profile for a second population of living cells.

In certain embodiments, the first VLP protein is specific to the first population of cells and the second VLP protein is specific to the second population of cells. Optionally, the first VLP protein is the VLP producing protein encoded by the first nucleic acid and/or the second VLP protein is the VLP producing protein encoded by the second nucleic acid.

In some embodiments, the binding of isolating step (e) is performed using an antibody and/or the binding of isolating step (g) is performed using an antibody.

In another embodiment, the first VLP protein and/or the second VLP protein is tagged. Optionally, the tag is an epitope tag.

An additional aspect of the disclosure provides a method for assessing a test compound for efficacy and/or toxicity in living cells, the method involving: (a) providing a population of living cells; (b) introducing a nucleic acid sequence encoding for a VLP producing protein to the living cells, where introduction of the nucleic acid sequence encoding for the VLP producing protein is sufficient to induce budding of VLPs from the living cells; (c) contacting the living cells with a test compound; (d) isolating VLPs produced by the living cells via binding of a VLP protein; and (e) obtaining analyte profile information from the isolated VLPs, where the analyte profile information indicates the efficacy and/or toxicity of the test compound, thereby assessing a test compound for efficacy and/or toxicity in living cells. Another aspect of the instant disclosure provides a method for assessing a test compound for efficacy and/or toxicity in living cells, the method involving: (a) providing a population of living cells; (b) introducing a first nucleic acid sequence harboring a nucleic acid sequence encoding for a VLP producing protein and a second nucleic acid sequence harboring a nucleic acid sequence encoding for an epitope-tagged viral surface protein to the living cells, where introduction of the nucleic acid sequence encoding for a VLP producing protein is sufficient to induce budding of VLPs from the living cells; (c) contacting the living cells with a test compound; (d) isolating VLPs produced by the living cells via binding of the epitope-tagged viral surface protein; and (e) obtaining analyte profile information from the isolated VLPs, where the analyte profile information indicates the efficacy and/or toxicity of the test compound, thereby assessing a test compound for efficacy and/or toxicity in living cells.

### Definitions

As used herein, the term “virus like particle (VLP) producing protein” refers to any protein capable of prompting a mammalian cell harboring such a VLP producing protein to produce a VLP. In certain aspects, a VLP producing protein can refer to a single, exogenous protein capable of inducing VLP formation (e.g., retroviral gag protein), or a VLP producing protein may act in concert with other VLP producing proteins to achieve VLP formation.

The term “epitope-tagged viral surface protein” as used herein refers to a protein of a virus for which at least a portion of the protein has surface exposure, and to which an epitope tag is attached. In certain aspects an “epitope-tagged viral surface protein” is an epitope tagged viral envelope protein (e.g., VSV-G).

The term “fusion protein” as used herein refers to an engineered polypeptide that combines sequence elements excerpted from two or more other proteins, optionally from two or more naturally-occurring proteins.

The terms “transfect,” “transfects,” “transfecting” and “transfection” as used herein refer to the delivery of nucleic acids (usually DNA or RNA) to the cytoplasm or nucleus of cells, e.g., through the use of cationic lipid vehicle(s) and/or by means of electroporation, or other art-recognized means of transfection.

The term “transduction,” as used herein refers to the delivery of nucleic acids (usually DNA or RNA) to the cytoplasm or nucleus of cells through the use of viral delivery, e.g., via lentiviral delivery vectors/plasmids, or other art-recognized means of transduction.

The term “plasmid” as used herein refers to a construction comprised of genetic material designed to direct transformation of a targeted cell. The plasmid consist of a plasmid backbone. A “plasmid backbone” as used herein contains multiple genetic elements positional and sequentially oriented with other necessary genetic elements such that the nucleic acid in a nucleic acid cassette can be transcribed and when necessary translated in the transfected or transduced cells. The term plasmid as used herein can refer to nucleic acid, e.g., DNA derived from a plasmid vector, cosmid, phagemid or bacteriophage, into which one or more fragments of nucleic acid may be inserted or cloned which encode for particular genes

A “viral vector” as used herein is one that is physically incorporated in a viral particle by the inclusion of a portion of a viral genome within the vector, e.g., a packaging signal, and is not merely DNA or a located gene taken from a portion of a viral nucleic acid. Thus, while a portion of a viral genome can be present in a plasmid of the present disclosure, that portion does not cause incorporation of the plasmid into a viral particle and thus is unable to produce an infective viral particle.

As used herein, the term “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

As used herein, the term “integrating vector” refers to a vector whose integration or insertion into a nucleic acid (e.g., a chromosome) is accomplished via an integrase. Examples of “integrating vectors” include, but are not limited to, retroviral vectors, transposons, and adeno associated virus vectors.

As used herein, the term “integrated” refers to a vector that is stably inserted into the genome (i.e., into a chromosome) of a host cell.

As used herein, the term “genome” refers to the genetic material (e.g., chromosomes) of an organism.

As used herein, the term “exogenous gene” refers to a gene that is not naturally present in a host organism or cell, or is artificially introduced into a host organism or cell.

The term “gene” refers to a nucleic acid (e.g., DNA or RNA) sequence that comprises coding sequences necessary for the production of a polypeptide or precursor (e.g., proinsulin). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain. The DNA or RNA sequence thus codes for the amino acid sequence.

As used herein, the term “variant,” when used in reference to a protein, refers to proteins encoded by partially homologous nucleic acids so that the amino acid sequence of the proteins varies. As used herein, the term “variant” encompasses proteins encoded by homologous genes having both conservative and nonconservative amino acid substitutions that do not result in a change in protein function, as well as proteins encoded by homologous genes having amino acid substitutions that cause decreased (e.g., null mutations) protein function or increased protein function.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “regulatory element” refers to a genetic element which controls some aspect of the expression of nucleic acid sequences. For example, a promoter is a regulatory element that facilitates the initiation of transcription of an operably linked coding region. Other regulatory elements are splicing signals, polyadenylation signals, termination signals, RNA export elements, internal ribosome entry sites, etc.

Transcriptional control signals in eukaryotes comprise “promoter” and “enhancer” elements. Promoters and enhancers consist of short arrays of DNA sequences that interact specifically with cellular proteins involved in transcription (Maniatis et al., Science 236:1237 [1987]). Promoter and enhancer elements have been isolated from a variety of eukaryotic sources including genes in yeast, insect and mammalian cells, and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see, Voss et al., Trends Biochem. Sci., 11:287 [1986]; and Maniatis et al., supra). For example, the SV40 early gene enhancer is very active in a wide variety of cell types from many mammalian species and has been widely used for the expression of proteins in mammalian cells (Dijkema et al, EMBO J. 4:761 [1985]). Two other examples of promoter/enhancer elements active in a broad range of mammalian cell types are those from the human elongation factor 1α gene (Uetsuki et al., J. Biol. Chem., 264:5791 [1989]; Kim et al., Gene 91:217 [1990]; and Mizushima and Nagata, Nuc. Acids. Res., 18:5322 [1990]) and the long terminal repeats of the Rous sarcoma virus (Gorman et al., Proc. Natl. Acad. Sci. USA 79:6777 [1982]) and the human cytomegalovirus (Boshart et al., Cell 41:521 [1985]).

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques such as cloning and recombination) such that transcription of that gene is directed by the linked enhancer/promoter.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, etc.). In contrast, a “regulatable” promoter is one which is capable of directing a level of transcription of an operably linked nucleic acid sequence in the presence of a stimulus (e.g., heat shock, chemicals, etc.) which is different from the level of transcription of the operably linked nucleic acid sequence in the absence of the stimulus. Certain promoters are also known in the art to impart tissue-specificity and/or temporal/developmental specificity to expression of a nucleic acid sequence under control of such a promoter.

Eukaryotic expression vectors may also contain “viral replicons” or “viral origins of replication.” Viral replicons are viral DNA sequences that allow for the extrachromosomal replication of a vector in a host cell expressing the appropriate replication factors. Vectors that contain either the SV40 or polyoma virus origin of replication replicate to high “copy number” (up to 104 copies/cell) in cells that express the appropriate viral T antigen. Vectors that contain the replicons from bovine papillomavirus or Epstein-Barr virus replicate extrachromosomally at “low copy number” (^{˜}100 copies/cell). However, it is not intended that expression vectors be limited to any particular viral origin of replication.

As used herein, the term “retrovirus” refers to a retroviral particle which is capable of entering a cell (i.e., the particle contains a membrane-associated protein such as an envelope protein or a viral G glycoprotein which can bind to the host cell surface and facilitate entry of the viral particle into the cytoplasm of the host cell) and integrating the retroviral genome (as a double-stranded provirus) into the genome of the host cell. The term “retrovirus” encompasses Oncovirinae (e.g., Moloney murine leukemia virus (MoMLV, also recited as simply “MLV” herein), Moloney murine sarcoma virus (MoMSV), and Mouse mammary tumor virus (MMTV), Spumavirinae, and Lentivirinae (e.g., Human immunodeficiency virus, Simian immunodeficiency virus, Equine infection anemia virus, and Caprine arthritis-encephalitis virus; See, e.g., U.S. Pat. Nos. 5,994,136 and 6,013,516, both of which are incorporated herein by reference).

As used herein, the term “retroviral vector” refers to a retrovirus that has been modified to express a gene of interest. Retroviral vectors can be used to transfer genes efficiently into host cells by exploiting the viral infectious process. Foreign or heterologous genes cloned (i.e., inserted using molecular biological techniques) into the retroviral genome can be delivered efficiently to host cells which are susceptible to infection by the retrovirus.

The term “Rhabdoviridae” refers to a family of enveloped RNA viruses that infect animals, including humans, and plants. The Rhabdoviridae family encompasses the genus Vesiculovirus which includes vesicular stomatitis virus (VSV), Cocal virus, Piry virus, Chandipura virus, and Spring viremia of carp virus (sequences encoding the Spring viremia of carp virus are available under GenBank accession number U18101). The G proteins of viruses in the Vesiculovirus genera are virally-encoded integral membrane proteins that form externally projecting homotrimeric spike glycoproteins complexes that are required for receptor binding and membrane fusion. The G proteins of viruses in the Vesiculovirus genera have a covalently bound palmititic acid (C16) moiety. The amino acid sequences of the G proteins from the Vesiculoviruses are fairly well conserved. For example, the Piry virus G protein share about 38% identity and about 55% similarity with the VSV G proteins (several strains of VSV are known, e.g., Indiana, New Jersey, Orsay, San Juan, etc., and their G proteins are highly homologous). The Chandipura virus G protein and the VSV G proteins share about 37% identity and 52% similarity. Given the high degree of conservation (amino acid sequence) and the related functional characteristics (e.g., binding of the virus to the host cell and fusion of membranes, including syncytia formation) of the G proteins of the Vesiculoviruses, the G proteins from non-VSV Vesiculoviruses may be used in place of the VSV G protein for the pseudotyping of viral particles. The G proteins of the Lyssa viruses (another genera within the Rhabdoviridae family) also share a fair degree of conservation with the VSV G proteins and function in a similar manner (e.g., mediate fusion of membranes) and therefore may be used in place of the VSV G protein for the pseudotyping of viral particles. The Lyssa viruses include the Mokola virus and the Rabies viruses (several strains of Rabies virus are known and their G proteins have been cloned and sequenced). The Mokola virus G protein shares stretches of homology (particularly over the extracellular and transmembrane domains) with the VSV G proteins which show about 31% identity and 48% similarity with the VSV G proteins. Preferred G proteins share at least 25% identity, preferably at least 30% identity and most preferably at least 35% identity with the VSV G proteins. The VSV G protein from which New Jersey strain (the sequence of this G protein is provided in GenBank accession numbers M27165 and M21557) is employed as the reference VSV G protein.

As used herein, the term “lentivirus vector” refers to retroviral vectors derived from the Lentiviridae family (e.g., human immunodeficiency virus, simian immunodeficiency virus, equine infectious anemia virus, and caprine arthritis-encephalitis virus) that are capable of integrating into non-dividing cells (See, e.g., U.S. Pat. Nos. 5,994,136 and 6,013,516, both of which are incorporated herein by reference).

As used herein, the term “adeno-associated virus (AAV) vector” refers to a vector derived from an adeno-associated virus serotype, including without limitation, AAV-1, AAV-2, AAV-3, AAV-4, AAV-5, AAVX7, etc. AAV vectors can have one or more of the AAV wild-type genes deleted in whole or part, preferably the rep and/or cap genes, but retain functional flanking ITR sequences.

As used herein the term, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

As used herein, the term “clonally derived” refers to a cell line that it derived from a single cell.

As used herein, the term “non-clonally derived” refers to a cell line that is derived from more than one cell.

As used herein, the term “passage” refers to the process of diluting a culture of cells that has grown to a particular density or confluency (e.g., 70% or 80% confluent), and then allowing the diluted cells to regrow to the particular density or confluency desired (e.g., by replating the cells or establishing a new roller bottle culture with the cells.

As used herein, the term “stable,” when used in reference to genome, refers to the stable maintenance of the information content of the genome from one generation to the next, or, in the particular case of a cell line, from one passage to the next. Accordingly, a genome is considered to be stable if no gross changes occur in the genome (e.g., a gene is deleted or a chromosomal translocation occurs). The term “stable” does not exclude subtle changes that may occur to the genome such as point mutations.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “host cell” refers to any eukaryotic cell (e.g., mammalian cells, avian cells, amphibian cells, plant cells, fish cells, and insect cells), whether located in vitro or in vivo.

As used herein, the term “next-generation sequencing” or “NGS” can refer to sequencing technologies that have the capacity to sequence polynucleotides at speeds that were unprecedented using conventional sequencing methods (e.g., standard Sanger or Maxam-Gilbert sequencing methods). These unprecedented speeds are achieved by performing and reading out thousands to millions of sequencing reactions in parallel. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (ion Torrent); and DNA nanoball sequencing (Complete Genomics). Descriptions of certain NGS platforms can be found in the following: Shendure, er al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3):333-43; and Zhang et al., “The impact of next-generation sequencing on genomics”, J Genet Genomics, 201, 38(3): 95-109.

The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

By “agent” is meant any small compound (e.g., small molecule), antibody, nucleic acid molecule, or polypeptide, or fragments thereof or cellular therapeutics such as allogeneic transplantation and/or CART-cell therapy.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, melanoma and ovarian cancer (e.g., cystadenocarcinoma, ovarian embryonal carcinoma, ovarian adenocarcinoma), with ovarian cancer specifically including clear cell ovarian cancer. Additional exemplary cancers include, but are not limited to, colorectal cancer (e.g., colon cancer, rectal cancer, colorectal adenocarcinoma), endometrial cancer (e.g., uterine cancer, uterine sarcoma), esophageal cancer (e.g., adenocarcinoma of the esophagus, Barrett's adenocarcinoma), and gastric cancer (e.g., stomach adenocarcinoma (STAD)), including, e.g., colon adenocarcinoma (COAD), oesophageal carcinoma (ESCA), rectal adenocarcinoma (READ) and uterine corpus endometrial carcinoma (UCEC). Other exemplary forms of cancer include, but are not limited to, diffuse large B-cell lymphoma (DLBCL), as well as the broader class of lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above, hematopoietic cancers (e.g., myeloid malignancies (e.g., acute myeloid leukemia (AML) (e.g., B-cell AML, T-cell AML), myelodysplastic syndrome, myeloproliferative neoplasm, chronic myelomonocytic leukemia (CMML) and chronic myelogenous leukemia (CML) (e.g., B-cell CML, T-cell CML)) and lymphocytic leukemia such as acute lymphocytic leukemia (ALL) (e.g., B-cell ALL, T-cell ALL) and chronic lymphocytic leukemia (CLL) (e.g., B-cell CLL, T-cell CLL)); brain cancer (e.g., meningioma, glioblastomas, glioma (e.g., astrocytoma, oligodendroglioma), medulloblastoma); lung cancer (e.g., bronchogenic carcinoma, small cell lung cancer (SCLC), non-small cell lung cancer (NSCLC), adenocarcinoma of the lung); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); bronchus cancer; carcinoid tumor; cervical cancer (e.g., cervical adenocarcinoma); choriocarcinoma; chordoma; craniopharyngioma; connective tissue cancer; epithelial carcinoma; ependymoma; endotheliosarcoma (e.g., Kaposi's sarcoma, multiple idiopathic hemorrhagic sarcoma); Ewing's sarcoma; ocular cancer (e.g., intraocular melanoma, retinoblastoma); familiar hypereosinophilia; gall bladder cancer; gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

By “control” or “reference” is meant a standard of comparison. Methods to select and test control samples are within the ability of those in the art. Determination of statistical significance is within the ability of those skilled in the art, e.g., the number of standard deviations from the mean that constitute a positive result.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the invention solely to the specific embodiments described, may best be understood in conjunction with the accompanying drawings.

FIGS. 1A to 1F display four schematic diagrams, a picture, and a graph, respectively, depicting an overview of self-reporting technology according to exemplary embodiments of the disclosure. FIG. 1A is a diagram showing that Virus-like Particles (VLPs) generated by endogenous or ectopic expression of gag or gag-like proteins allow for export of cellular contents from living cells. FIG. 1B is a diagram that is expanded from the inset square in FIG. 1A, which shows that proteins, lipids, metabolites, small molecules, RNA and/or DNA can be exported via VLPs. FIG. 1C is a diagram showing that VLPs are comprised of viral capsid proteins, with a core carrying analytes of interest. FIG. 1D is a diagram showing that VLPs can be collected from the same culture over time, and purified with immunoprecipitation (IP)—optionally via IP of cell line-specific and/or virion-specific tags—centrifugation, concentration via molecular weight cutoff filters, gradients, crowding agents (such as PEG), or a combination of the mentioned methods. FIG. 1E is a picture showing that if RNA is the analyte of interest, RNAseq libraries can be generated and sequenced (e.g., with Next Generation Sequencing (NGS) technology. FIG. 1F is a graph showing that time series measurements can be analyzed from the same biological samples, to provide longitudinal information about the same biological sample throughout time.

FIGS. 2A to 2D display a Western Blot, a dot plot, a schematic, and a dot plot, respectively, showing that flag-mediated immunoprecipitation of VLPs enabled clean RNAseq library construction with minimal background according to exemplary embodiments of the disclosure. FIG. 2A shows a Western Blot that was performed to measure production of Gag protein in lysate, VLP generation (measured by Gag protein in supernatant), and envelope-based immunoprecipitation (measured by Gag protein detected after a flag immunoprecipitation). In this experiment; 293T cells were transfected with MLV gag, VSV-g (envelope), flag-VSV-g (flag-envelope) and/or pUC19 (negative control). FIG. 2B shows dot plot data of supernatants obtained from conditions where MLV gag was transfected, which demonstrated that the instant approach generated high quality RNAseq libraries, as measured by genes detected. FIG. 2C is a schematic showing a VLP with envelope glycoproteins, such as VSV-g (adapted electron cryo tomograph from Riedel et al., J Struct Biol. 2017). FIG. 2D shows dot plot data in which VLPs labeled with flag-VSV-g were demonstrated to have generated high quality RNAseq libraries after flag immunoprecipitation, as measured by genes detected. Background from VLPs without flag-labeled envelopes was identified to be negligible.

FIG. 3 provides a schematic diagram (top) and a graph (bottom) showing that affinity-tagged VLPs generated high quality RNAseq libraries that exhibited low background. Affinity-tag based immunoprecipitation (IP) was conducted to determine whether RNA could be captured and selectively purified from pseudotyped (flag-VSV-g+, or HA-VSV-g+) virus-like particles (VLPs). Utilizing two different cell types (293T and HT1080) and two different affinity tagged envelopes (flag-VSV-g, HA-VSV-g), high quality RNAseq libraries (quantified by genes detected) were generated when each supernatant was put through the matching immunoprecipitation. Conversely, poor quality RNAseq libraries (quantified by genes detected) were generated when each supernatant was put through an incorrect immunoprecipitation. These data were collected from cell-lines with stable, single-copy expression of gag and an affinity-labeled VSV-g, integrated via lentivirus.

FIG. 4 provides a schematic diagram (top) and a graph (bottom) showing that affinity-tagged envelopes can be used to non-destructively classify two distinct, exporting (gag+) cell-types from living co-culture. 293T and HT1080 VLP-producing cell lines (gag+) were co-cultured and supernatants were collected and processed via IP. Supernatants processed via flag-IP generated RNAseq libraries that classified as 293T cells, demonstrating that the flag-IP captured VLPs were indeed from 293 Ts. Similarly, supernatants processed via HA-IP generated RNAseq libraries that classified as HT1080 cells, demonstrating that the HA-IP captured VLPs were indeed from HT1080s. These data were collected from cell-lines with stable, single-copy expression of gag and an affinity-labelled VSV-g, integrated via lentivirus.

FIG. 5 is a series of graphs showing that envelope-based multiplexing for live-cell monitoring is quantitative. RNAseq data show that quantitative transcriptional information can be measured from live-cell co-cultures via purification of affinity-tagged VLPs via immunoprecipitation.

### DETAILED DESCRIPTION OF THE INVENTION

The current disclosure relates, at least in part, to the identification of compositions and methods capable of inducing living cells to produce virus-like particles (VLPs) and allowing for highly specific isolation of such VLPs, which thereby enables real-time and/or time course assessment of VLP-captured analytes obtained from targeted living cells. Certain aspects of the instant disclosure feature introduction of a nucleic acid(s) encoding for (i) a VLP producing protein and (ii) an epitope-tagged viral surface protein into a living cell, which induces the living cell to produce VLPs that can then be readily isolated (e.g., by immunoprecipitation) via binding of the epitope tag. Such VLPs capture analytes from the living cells at the time of budding, meaning that assessment of VLP-encapsulated analytes (e.g., RNA) can provide a profile of such analytes in living cells over a time course, without harming the living cells (beyond any harm that might be done to the cells during transfection/transduction of the cells with the nucleic acid(s) encoding for the VLP producing protein and the epitope-tagged viral surface protein). When RNA is assessed as the VLP analyte, transcriptome profiling of the living cells can be performed in real time and/or over a time course while leaving the cells intact, which provides a significant benefit over other art-recognized transcriptome profiling methods, in contexts where survival of the cells for which transcriptome profiling is performed is advantageous.

In related aspects, distinct epitope tags can be employed to distinguish between different cell populations during analyte profiling (e.g., expression profiling) of living cells, even when such cell populations are mixed, which provides certain advantages over other art-recognized methods of analyte profiling.

Various expressly contemplated components of certain compositions and methods of the instant disclosure are considered in additional detail below.

### Virus Like Particles (VLPs)

In certain aspects, the present disclosure provides compositions and methods that related to isolating and analyzing virus-like particles (VLPs) that present affinity-tagged envelope proteins. Virus-like particles (VLPs) are artificial protein structures that exhibit overall structure similar to their corresponding native viruses. VLPs resemble viruses in their self-assembly property but lack original infectious ability due to the genome modifications. VLPs can be symmetrically built from hundreds to thousands of coat proteins, which can be genetically engineered to present a regular arrangement of epitopes on the desired positions of the outer surface. Compared with monomeric or oligomeric protein carriers, VLPs are able to provide not only a higher density of foreign proteins per particle but also support a distinctive three-dimensional conformation, which, without wishing to be bound by theory, has been described as especially important for the presentation of conformational epitopes. To date, VLPs have been recognized as one of the most promising and extensively studied molecular carriers or nanoparticles, for a variety of applications. (Zeltins et al. Molecular biotechnology, 53: 92-107).

Viral Gag Proteins

Certain aspects of the present disclosure relate to compositions and methods for isolating and analyzing VLPs generated via endogenous or ectopic expression of Gag (Group-specific antigen) or Gag-like proteins, which allow for export of cellular contents from living cells. The Gag polyprotein is a protein constructed from the nucleotide sequence of a retrovirus's RNA sequence. Gag polyproteins are used in the viral replication cycle of a retrovirus. The assembly and release of retrovirus particles from the cell membrane is directed by the Gag polyprotein. Utilizing methods of protein sequencing, scientists have begun to understand how these proteins can interact with the host cells and prevent infection. To date, no approach has been exploited in determining antiviral therapy utilizing Gag proteins due to the lack of knowledge concerning the structures and interactions responsible for assembly. The sequence of the Gag protein depends upon Gag-nucleic acid interactions. Nucleic acid sequences as short as 20-40 nucleotides can support VLP assembly in vitro. Since the Gag protein is the fundamental building block of the retrovirus particles, one expression of the gene into the Gag protein is sufficient to prompt replication of VLPs. The Gag protein itself has multiple domains within the complex. This multi-domain of the Gag protein participates in interactions with lipids in the plasma membrane, RNAs, and other Gag molecules. Gag proteins undergo conformational changes during virus particle assembly. In a Gag protein there is an N-terminal matrix domain (MA) and a C-terminal nucleocapsid domain (NC). Although both domains are positively charged and have affinities for negatively charged ions, the matrix domain has a high affinity for lipids due to the presence of phosphatidyl inositol bisphosphate, while the nucleocapsid domain has a high affinity for nucleic acids. Without wishing to be bound by theory, it is believed that this affinity allows for the Gag protein to become rod-like upon entering the plasma membrane of the nucleous that contains nucleic acids. (Rein et al. Trends Biochem Sci. 36(7): 373-80).

Exemplary Gag and Gag-like proteins include, but are not limited to, a retrovirus gag protein (e.g., a HIV Gag viral protein (e.g., HIV-1 NL43 Gag (GenBank serial number AAA44987), a simian immunodeficiency virus (SIV) Gag viral protein (e.g., SIVmac239 Gag (GenBank serial number CAA68379)), or a murine leukemia virus (MLV) Gag viral protein, such as GenBank serial number S70394):

MLV Gag viral protein
(SEQ ID NO: 1)
MGQAVTTPLSLTLDHWKDVERTAHNLSVEVRKRRWVTFCSAEWPTFNVGW
PRDGTFNPDIITQVKIKVFSPGPHGHPDQVPYIVTWEAIAVDPPPWVRPF
VHPKPPLSLPPSAPSLPPEPPLSTPPQSSLYPALTSPLNTKPRPQVLPDS
GGPLIDLLTEDPPPYRDPGPPSPDGNGDSGEVAPTEGAPDPSPMVSRLRG
RKEPPVADSTTSQAFPLRLGGNGQYQYWPFSSSDLYNWKNNNPSFSEDPA
KLTALIESVLLTHQPTWDDCQQLLGTLLTGEEKQRVLLEARKAVRGEDGR
PTQLPNDINDAFPLERPDWDYNTQRGRNHLVHYRQLLLAGLQNAGRSPTN
LAKVKGITQGPNESPSAFLERLKEAYRRYTPYDPEDPGQETNVAMSFIWQ
SAPDIGRKLERLEDLKSKTLGDLVREAEKIFNKRETPEEREERIRRETEE
KEERRRAEDVQREKERDRRRHREMSKLLATVVSGQRQDRQGGERRRPQLD
HDQCAYCKEKGHWARDCPKKPRGPRGPRPQASLLTLDD

Additional exemplary Gag and Gag-like proteins include, but are not limited to, a retrovirus matrix protein, a rhabdovirus matrix protein M protein (e.g., a vesicular stomatis virus (VSV) M protein (GenBank serial number NP041714)), a filovirus viral core protein (e.g., an Ebola VP40 viral protein (e.g., Ebola virus VP40 (GenBank serial number AAN37506))), a Rift Valley Fever virus N protein (e.g., RVFV N Protein (GenBank serial number NP049344)), a coronavirus M, E and NP protein (e.g., GenBank serial number NP040838 for NP protein, NP 040835 for M protein, CAC39303 for E protein of Avian Infections Bronchitis Virus and NP828854 for E protein of the SARS virus)), a bunyavirus N protein (GenBank serial number AAA47114)), an influenza M1 protein, a paramyxovirus M protein, an arenavirus Z protein (e.g., a Lassa Fever Virus Z protein), and combinations thereof. Appropriate surface glycoproteins and/or viral RNA may be included to form the VLP.

In some embodiments, nonenveloped virus capsid proteins can be used. Examples of non-enveloped viruses include those of the virus families Adenoviridae, Papovaviridae, Parvoviridae, and Anelloviridae.

Without wishing to be bound by theory, Gag proteins are believed to be the core structural proteins of a retrovirus.

Retroviruses

Retroviruses refer to a family of viruses which have RNA and reverse transcriptase (RNA-dependent DNA polymerase), of which the latter is essential to the first stage of its self-replication for synthesizing complementary DNA on the base of template RNA of the virus. Retroviruses can be categorized into Orthoretrovirinae (includes oncoviruses and lentiviruses) and Spumaretrovirinae. The oncoviruses are thus termed because they can be associated with cancers and malignant infections. There may be mentioned, for example, leukemogenic viruses such as the avian leukemia virus (ALV), the murine leukemia virus (MULV), also called Moloney virus or simply MLV at some instances herein, the Abelson leukemia virus, the murine mammary tumor virus, the Mason-Pfizer monkey virus (or MPMV), the feline leukemia virus (FELV), human leukemia viruses such as HTLV1 (also, named HTLV-I) and HTLV2 (also named HTLV-Π), the simian leukemia virus or STLV, the bovine leukemia virus or BLV, the primate type D oncoviruses, the type B oncoviruses which are inducers of mammary tumors, or oncoviruses which cause a rapid cancer (such as the Rous sarcoma virus or RSV).

Although the term “oncovirus” is still commonly used, other terms can also be used such as Alpharetrovirus for avian leukosis virus and Rous sarcoma virus; Betaretrovirus for mouse mammary tumor virus; Gammaretrovirus for murine leukemia virus and feline leukemia virus; Deltaretrovirus for bovine leukemia virus and human T-lymphotropic virus; and Epsilonretrovirus for Walleye dermal sarcoma virus. The lentiviruses, such as Human Immunodeficiency Virus (HIV, also known as HTLV-III or LAV for lymphotrophic adenovirus and which can be distinguished within HTV-1 and HTV-2), are thus named because they are responsible for slow-progressing pathological conditions which very frequently involve immunosuppressive phenomena, including AIDS. Among the lentiviruses, the visna/maedi virus (or MW/Visna), equine infectious anemia virus (EIAV), caprine arthritis encephalitis virus (CAEV), simian immunodeficiency virus (SIV) can also be cited (See, e.g., WO2015001518A1, which is incorporated herein by reference).

The spumaviruses manifest fairly low specificity for a given cell type or a given species, and they are sometimes associated with immunosuppressive phenomena; that is the case, for example, for the simian foamy virus (or SFV), also named chimpanzee simian virus, the human foamy virus (or HFV), bovine syncytial virus (or BSV), feline syncytial virus (FSV) and the feline immunodeficiency virus.

Adeno-Associated Viruses (AAV)

Adeno-associated viruses (AAV) are small (about 20 nm) nonenveloped icosahedric ssDNA viruses, which depend on helper viruses (e.g., adenovirus or herpes simplex virus) for replication. To date, nine human serotypes have been characterized. About 80% of the population has detectable levels of anti-AAV antibodies, but there is no discernable pathology association with this virus. This fact and the ability of AAV to mediate transgene integration into a specific site in the human genome have made it an important candidate for use in gene therapy. The resulting knowledge about capsid structure and tolerance for peptide insertions has been described for use in the design of genome-free AAV-like particles (AAVLPs) as a novel high-density system for peptide vaccines. (Manzano-Szalai et al. Viral Immunol. 2014 Nov. 1; 27(9): 438-448). It is expressly contemplated herein that AAV VLPs can be used in the compositions and methods of the instant disclosure.

Isolation and Purification of VLPs

In certain aspects, the compositions and methods of the present disclosure relate to isolating and analyzing virus-like particles (VLPs), optionally those presenting cell line specific affinity-tagged envelope proteins. VLPs of the instant disclosure can be isolated and purified by many methods including, but not limited to, immunoprecipitation (IP), gradient centrifugation, chromatography (e.g., gel filtration chromatography), assays, fractionation, quantitation, and electrophoresis. Certain aspects of the instant disclosure present immunoprecipitation that utilizes an affinity tagged viral envelope protein for VLP isolation and ultimate compilation of a clean RNA sequence library possessing minimal background. Immunoprecipitation (IP) is a method used to isolate a specific antigen from a mixture, using the antigen-antibody interaction. Antigens isolated by IP are typically analyzed by SDS-PAGE or Western blotting. In IP, an antibody is added first to a mixture containing an antigen, and incubated to allow antigen-antibody complexes to form. Subsequently, the antigen-antibody complexes are incubated with an immobilized antibody against the primary antibody (secondary antibody) or with protein AIG-coated beads to allow them to absorb the complexes. The beads are then thoroughly washed, and the antigen is eluted from the beads by an acidic solution or SDS. If suitable antibody is not available, the target molecule can be fused to a protein tag by recombinant DNA techniques, and IP can proceed using an antibody to the tag (pull-down assay).

In certain aspects, the present disclosure relates to compositions and methods for isolating and analyzing virus-like particles (VLPs) having cell line specific affinity-tagged envelopes (such as FLAG (epitope tag) tagged viral envelope (VSV-g)). Epitope tagging is a procedure whereby a short amino acid sequence recognized by a preexisting antibody is attached to a protein under study to allow its recognition by the antibody in a variety of in vitro or in vivo settings. A primary advantage of epitope tagging is that the time and expense associated with generating and characterizing antibodies against multiple proteins is obviated. However, epitope tagging offers a number of additional advantages such as: it allows tracking of closely related proteins without fear of spurious results resulting from cross-reactive antibodies; the intracellular location of epitope-tagged proteins can be identified in immunofluorescence experiments in a similarly well-controlled manner, without fear of cross-reactivity with the endogenous protein; the epitope-tagging approach can be particularly useful for discriminating among otherwise similar gene products that cannot be distinguished using conventional antibodies. For example, epitope tagging permits discrimination of individual members of closely related protein families or the identification of in vitro-mutagenized variants in the context of endogenous wild-type protein(s).

As specifically exemplified herein, a Vesicular Stomatitis Virus (VSV) glycoprotein was epitope tagged to improve targeted isolation of VLPs. An exemplary sequence for VSV glycoprotein is:

VSV-G Envelope Protein (VSV Glycoprotein; SEQ ID
NO: 2) precursor:
MKCLLYLAFLFIGVNCKFTIVFPHNQKGNWKNVPSNYHYCPSSSDLNWHN
DLIGTALQVKMPKSHKAIQADGWMCHASKWVTTCDFRWYGPKYITHSIRS
FTPSVEQCKESIEQTKQGTWLNPGFPPQSCGYATVTDAEAVIVQVTPHHV
LVDEYTGEWVDSQFINGKCSNYICPTVHNSTTWHSDYKVKGLCDSNLISM
DITFFSEDGELSSLGKEGTGFRSNYFAYETGGKACKMQYCKHWGVRLPSG
VWFEMADKDLFAAARFPECPEGSSISAPSQTSVDVSLIQDVERILDYSLC
QETWSKIRAGLPISPVDLSYLAPKNPGTGPAFTIINGTLKYFETRYIRVD
IAAPILSRMVGMISGTTTERELWDDWAPYEDVEIGPNGVLRTSSGYKFPL
YMIGHGMLDSDLHLSSKAQVFEHPHIQDAASQLPDDESLFFGDTGLSKNP
IELVEGWFSSWKSSIASFFFIIGLIIGLFLVLRVGIHLCIKLKHTKKRQI
YTDIEMNRLGK

The initial 16 amino acids of the VSV-G envelope protein precursor are removed during processing, resulting in the mature form of the VSV-G Envelope Protein (VSV Glycoprotein, mature; SEQ ID NO: 21):

KFTIVFPHNQKGNWKNVPSNYHYCPSSSDLNWHNDLIGTALQVKMPKSHK
AIQADGWMCHASKWVTTCDFRWYGPKYITHSIRSFTPSVEQCKESIEQTK
QGTWLNPGFPPQSCGYATVTDAEAVIVQVTPHHVLVDEYTGEWVDSQFIN
GKCSNYICPTVHNSTTWHSDYKVKGLCDSNLISMDITFFSEDGELSSLGK
EGTGFRSNYFAYETGGKACKMQYCKHWGVRLPSGVWFEMADKDLFAAARF
PECPEGSSISAPSQTSVDVSLIQDVERILDYSLCQETWSKIRAGLPISPV
DLSYLAPKNPGTGPAFTIINGTLKYFETRYIRVDIAAPILSRMVGMISGT
TTERELWDDWAPYEDVEIGPNGVLRTSSGYKFPLYMIGHGMLDSDLHLSS
KAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK

In some embodiments, a mutagenized VSV-G protein is employed. Optionally, mutagenesis of VSV-G protein produces a VSV-G protein that prevents VLP uptake. Examples of such VSV-G protein mutations include K47 and R354 VSV-G mutants (see Nikolic et al. Nature Communications, volume 9, Article number: 1029 (2018)).

VSV-G K47A Mutant Envelope Protein
(VSV Glycoprotein, mature; SEQ ID NO: 22):
KFTIVFPHNQKGNWKNVPSNYHYCPSSSDLNWHNDLIGTALQVKMPASHK
AIQADGWMCHASKWVTTCDFRWYGPKYITHSIRSFTPSVEQCKESIEQTK
QGTWLNPGFPPQSCGYATVTDAEAVIVQVTPHHVLVDEYTGEWVDSQFIN
GKCSNYICPTVHNSTTWHSDYKVKGLCDSNLISMDITFFSEDGELSSLGK
EGTGFRSNYFAYETGGKACKMQYCKHWGVRLPSGVWFEMADKDLFAAARF
PECPEGSSISAPSQTSVDVSLIQDVERILDYSLCQETWSKIRAGLPISPV
DLSYLAPKNPGTGPAFTIINGTLKYFETRYIRVDIAAPILSRMVGMISGT
TTERELWDDWAPYEDVEIGPNGVLRTSSGYKFPLYMIGHGMLDSDLHLSS
KAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK
VSV-G R354A Mutant Envelope Protein
(VSV Glycoprotein, mature; SEQ ID NO: 23):
KFTIVFPHNQKGNWKNVPSNYHYCPSSSDLNWHNDLIGTALQVKMPKSHK
AIQADGWMCHASKWVTTCDFRWYGPKYITHSIRSFTPSVEQCKESIEQTK
QGTWLNPGFPPQSCGYATVTDAEAVIVQVTPHHVLVDEYTGEWVDSQFIN
GKCSNYICPTVHNSTTWHSDYKVKGLCDSNLISMDITFFSEDGELSSLGK
EGTGFRSNYFAYETGGKACKMQYCKHWGVRLPSGVWFEMADKDLFAAARF
PECPEGSSISAPSQTSVDVSLIQDVERILDYSLCQETWSKIRAGLPISPV
DLSYLAPKNPGTGPAFTIINGTLKYFETRYIRVDIAAPILSRMVGMISGT
TTEAELWDDWAPYEDVEIGPNGVLRTSSGYKFPLYMIGHGMLDSDLHLSS
KAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK
VSV-G K47A, R354A Mutant Envelope Protein
(VSV Glycoprotein, mature; SEQ ID NO: 28):
KFTIVFPHNQKGNWKNVPSNYHYCPSSSDLNWHNDLIGTALQVKMPASHK
AIQADGWMCHASKWVTTCDFRWYGPKYITHSIRSFTPSVEQCKESIEQTK
QGTWLNPGFPPQSCGYATVTDAEAVIVQVTPHHVLVDEYTGEWVDSQFIN
GKCSNYICPTVHNSTTWHSDYKVKGLCDSNLISMDITFFSEDGELSSLGK
EGTGFRSNYFAYETGGKACKMQYCKHWGVRLPSGVWFEMADKDLFAAARF
PECPEGSSISAPSQTSVDVSLIQDVERILDYSLCQETWSKIRAGLPISPV
DLSYLAPKNPGTGPAFTIINGTLKYFETRYIRVDIAAPILSRMVGMISGT
TTEAELWDDWAPYEDVEIGPNGVLRTSSGYKFPLYMIGHGMLDSDLHLSS
KAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK

In certain aspects, targeted molecules used in the isolation of VLPs via IP or other such technique are viral surface envelope glycoproteins, which can include, but are not limited to, a retrovirus glycoprotein (e.g., a human immunodeficiency virus (HIV) envelope glycoprotein (e.g., HIVSF162 envelope glycoprotein (GenBank serial number M65024)), a simian immunodeficiency virus (SIV) envelope glycoprotein (e.g., SIVmac239 envelope glycoprotein (GenBank serial number M33262)), a simian-human immunodeficiency virus (SHIV) envelope glycoprotein (e.g., SHIV-89.6p envelope glycoprotein (GenBank serial number U89134)), a feline immunodeficiency virus (FIV) envelope glycoprotein (e.g., feline immunodeficiency virus envelope glycoprotein (GenBank serial number L00607)), a feline leukemia virus envelope glycoprotein (e.g., feline leukemia virus envelope glycoprotein (GenBank serial number M12500)), a bovine immunodeficiency virus envelope glycoprotein (e.g., bovine immunodeficiency virus envelope glycoprotein (GenBank serial number NC001413)), a bovine leukemia virus envelope glycoprotein (GenBank serial number AF399703), an equine infectious anemia virus envelope glycoprotein (e.g., equine infectious anemia virus envelope glycoprotein (GenBank serial number NC001450)), a human T-cell leukemia virus envelope glycoprotein (e.g., human T-cell leukemia virus envelope glycoprotein (GenBank serial number AF0033817)), and a mouse mammary tumor virus envelope glycoprotein (MMTV)), a bunyavirus glycoprotein (e.g., a Rift Valley Fever virus (RVFV) glycoprotein, (e.g., RVFV envelope glycoprotein (GenBank serial number M11157))), an arenavirus glycoprotein (e.g., a Lassa fever virus glycoprotein (GenBank serial number AF333969))), a filovirus glycoprotein (e.g., an Ebola virus glycoprotein (GenBank serial number NC002549)), a corona virus glycoprotein (GenBank serial number SARS coronavirus spike protein AAP13567), an influenza virus glycoprotein (GenBank serial number V01085)), a paramyxovirus glycoprotein (GenBank serial number NC002728 for Nipah virus F and G proteins), a rhabdovirus glycoprotein (GenBank serial number NP049548)) (e.g., a Vesicular Stomatitis Virus (VSV) glycoprotein as exemplified), an alphavirus glycoprotein (GenBank serial number AAA48370 for Venezuelan equine encephalomyelitis (VEE)), a flavivirus glycoprotein (GenBank serial number NC001563 for West Nile virus) (e.g., a Hepatitis C Virus glycoprotein), a Herpes Virus glycoprotein (e.g., a cytomegalovirus glycoprotein), and combinations thereof.

In certain embodiments, nonenveloped capsid proteins can be used in the compositions and methods of the instant disclosure, such as capsid proteins from the virus families Adenoviridae, Papovaviridae, Parvoviridae, and Anelloviridae. In this context, the capsid can be mutagenized to prevent VLP uptake by neighboring cells, and an affinity tag can be optionally introduced to the capsid for purification. Also, the VLP can be purified with antibodies that bind to a non-tagged capsid.

In some embodiments, the VLPs naturally incorporate host membrane proteins, and VLPs can be purified by affinity-tagged host membrane proteins, or by using antibodies against host membrane proteins.

Exemplary epitope tags that can be attached to a targeted molecule can include, but are not limited to FLAG (DYKDDDDK; SEQ ID NO: 3), 6×His (HHHHHH; SEQ ID NO: 4), HA (YPYDVPDYA; SEQ ID NO: 5), c-myc (EQKLISEEDL; SEQ ID NO: 6), V5 tag (GKPIPNPLLGLDST; SEQ ID NO: 7), AU1 tag (DTYRYI; SEQ ID NO: 8), AU5 tag (TDFYLK; SEQ ID NO: 9), Glu-Glu tag (EYMPME; SEQ ID NO: 10), OLLAS (SGFANELGPRLMGK; SEQ ID NO: 11), T7 tag (MASMTGGQQMG; SEQ ID NO: 12), VSV-G tag (YTDIEMNRLGK; SEQ ID NO: 13), E-Tag (GAPVPYPDPLEPR; SEQ ID NO: 14), S-Tag (KETAAAKFERQHMDS; SEQ ID NO: 15), HSV tag (SQPELAPEDPED; SEQ ID NO: 16), KT3 tag (KPPTPPPEPET; SEQ ID NO: 17), TK15 tag, GST tag, Protein A tag, CD tag, Strep-Tag (WSHPQFEK; SEQ ID NO: 18), MBP tag, CBD tag, Avi tag (CGLNDIFEAQKIEWHE; SEQ ID NO: 19), CBP tag, TAP tag, and SF-TAP tag. It is noted that in certain aspects, the above-referenced VSV-G tag is excluded from the above-recited list of contemplated epitope tags for inclusion in the compositions and methods of the instant disclosure.

As also noted above, in certain embodiments, purification of non-tagged envelope proteins (for enveloped VLPs) and/or non-tagged capsid proteins (for nonenveloped VLPs) can be performed using antibodies and/or affinity-binding methods.

### Viral Transfection of Mammalian Cells

In certain aspects, the compositions and methods of the present disclosure relate to production of virus-like particles (VLPs) using viral vector-mediated transfection of nucleic acids that encode for VLP-inducing agents. Viral vectors have received much attention in recent years and have become powerful tools for gene delivery in vitro and in vivo. In cultured cells, viruses are primarily used to achieve stable genomic integration and an inducible expression of transgenes. In vivo, viruses are often the only viable option when aiming at efficiently introducing transgenes into specific cell types, as is needed, for instance, in gene therapy. Virus-mediated transfection, also known as transduction, offers a means to reach hard-to-transfect cell types for protein overexpression or knockdown, and it is the most commonly used method in clinical research. Adenoviral, oncoretroviral, and lentiviral vectors have been used extensively for gene delivery in mammalian cell culture and in vivo. Other well-known examples for viral gene transfer include baculovirus and vaccinia virus-based vectors. Any of these and other art-recognized viral gene transfer systems are contemplated for use in the context of the instant disclosure.

A typical transduction protocol involves engineering of the recombinant virus carrying the transgene, amplification of recombinant viral particles in a packaging cell line, purification and titration of amplified viral particles, and subsequent infection of the cells of interest. While the achieved transduction efficiencies in primary cells and cell lines are quite high (˜90-100%), only cells carrying the viral-specific receptor can be infected by the virus. It is also important to note that the packaging cell line used for viral amplification needs to be transfected with a non-viral transfection method

Suitable mammalian cells that can be used for viral transduction include, but are not limited to, primary cells and cell lines, where suitable cell lines include, but are not limited to, 293 cells, COS cells, HeLa cells, Vero cells, 3T3 mouse fibroblasts, C3H10T1/2 fibroblasts, CHO cells, and the like. Non-limiting examples of suitable host cells include, e.g., HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In certain embodiments, exemplary mammalian cells for viral transduction are primary rat cortical neurons and/or primary rat hippocampal neurons, optionally those obtained from microsurgically dissected tissue, e.g., from E18 Sprague Dawley Rat.

Viral vectors have been employed in the study of various models of diseases such as metabolic, cardiovascular, muscular, hematologic, ophthalmologic, and infectious diseases and different types of cancer. Viral vectors such a retroviruses, adenoviruses, and herpes simplex viruses have been used in animal models and clinical trials of diseases such as, but not limited to, anaplastic thyroid cancer, carcinoma, hepatocellular carcinoma, glioma, hemophilia, Alzheimer's disease, sensory neuropathy, acquired immunodeficiency syndrome (AIDS), melanoma, Huntington's disease, and glioblastoma (Lundstrom. Diseases. 6(2) 42).

### Neuronal Cell Disease Models

Animal modeling of human disease is a cornerstone to basic scientific studies of disease mechanisms and pre-clinical studies of potential therapies. Rapid progress in in vitro, in vivo, and ex vivo animal modeling has led to advancements in the understanding of fundamental disease mechanisms of many central nervous system (CNS) disorders, including but not limited to, initial cell death and later repair in stroke, motor and non-motor pathologies in Parkinson's disease, and axonal regeneration in peripheral and optic nerve injury, among many others. Ideally, animal modeling produces basic insights, new views of the human disease, and preclinical trials of novel therapies (Chesselet et al. Neurotherapeutics. 9(2): 241-244).

Many animal models have been used in the study of neurological disease such as rodents (rat and mice) and primates. The mouse model has been particularly studied extensively as a neurological disease model. The common house mouse (Mus musculus) has a genome with 97% homology to the human genome. Mouse models of neurological disorders can be usefully divided into whether or not the model is heritable. Human neurological disorders with a mutant gene component make ideal candidates for modelling via gene manipulation. It follows then that human neurological disorders with an identified underlying genetic component, for example Alzheimer's disease, have been extensively modelled using genetically manipulated mouse models. Alternatively an interesting neurological phenotype may be identified as a result of a spontaneous mutation in the wild type mouse population, for example the stargazer mouse. These spontaneous mutant mouse models are then bred to sustain the appropriate phenotype of interest. Clearly neurological disorders also have heritable traits that do not include mutant gene components but are well characterized risk factors for the disorder, for example the Apoe4 allele in AD. As these traits can be inherited from generation to generation they can also be included as heritable trait models. Mouse models that do not carry a heritable component are focused on replicating a phenotype characteristic of the relevant disorder. Those human disorders that do not have a defined genetic component, or in which a complex multi-gene interacting system is under investigation, are more readily modelled using non heritable mouse models that have an identified robust phenotype and are acquired by physical manipulation (Harper. BBA. 10: 785-795).

Where animal models are employed, in some embodiments, specificity can be achieved through delivery, such as via use of a pseudotyped virus that only infects neurons, or a subset of neurons, and/or passes the blood brain barrier. For example, the AAV-PHP.B2 capsid can be used (from www.nature.com/articles/nbt.3440) to generate AAV carrying the VLP-inducing and envelope transgenes. This AAV can be administered via IV, allowing for delivery of the transgenes to the brain. Cerebral spinal fluid (CSF) can then be harvested from the animal to measure transcriptomes in different structures in the brain (such as the hippocampus), as well as different cell types in the brain (such as glial cells).

Neuronal Tissue-Specific Promoters

In certain aspects, the compositions and methods of the present disclosure include components that impart tissue-specificity to formation of particular types of VLPs. In one exemplified embodiment, to successfully label VLPs from excitatory neurons, a CamKII promoter can be used to drive expression of both a VLP producing protein, such as MLV Gag, as well as a labeled envelope protein, such as FLAG-VSVG. Meanwhile, to successfully label VLPs from inhibitory neurons, a mDIx promoter can be used to drive expression of both a VLP producing protein such as MLV Gag, as well as a labeled envelope protein, such as HA-VSVG. Such a system allows for direct comparison in real-time (and across a time course) of excitatory neuron transcriptomes vs. inhibitory neuron transcriptomes, from living cells of each respective type, even in mixed culture. Examples of neural tissue-specific promoters that can be employed in the compositions and methods of the instant disclosure include, but are not limited to, mDIx, CamKII, Syn1, NSE, PDGF and Ta1.

Compound Screening in Model Systems

Model systems, including laboratory animals, microorganisms, and cell- and tissue-based systems, are central to the discovery and development of new and better drugs for the treatment of human disease. Model systems such as animal models are essential for translation of drug findings from bench to bedside. Hence, critical evaluation of the face and predictive validity of these models is important. Reversely, clinical bedside findings that were not predicted by animal testing should be back translated and used to refine the animal models. Design, execution and reporting of results from animal model systems help to make preclinical data more reproducible and translatable to the clinic. Design of an animal model strategy is part of the translational plan rather than (a) single experiment(s). Data from animal models are essential in predicting the clinical outcome for a specific drug in development. Review, standardization and refinement of animal models by disease expert groups helps to improve rigor of animal model testing. It is important that the applied animal models are validated fit-for-purpose according to stringent criteria and reproducible. During drug development fit-for-purpose animal models are key for success in clinical translation, financial investments and support from the government to develop, optimize, validate and run such translation tools are important. Over time, this will be of benefit for patients and healthcare institutions. Preclinical testing of a drug in an animal model is not a prerequisite for regulatory agencies before entering clinical trials, but does unquestionably provide valuable data on the expected clinical performance of the drug. Hence, testing in animal models is largely recommended from both a business and patient perspective. In addition, inclusion of safety parameters in animal models will help to build the required safety data package of drugs in development (Denayer et al. Translational Medicine. 2: 5-11).

It is herein expressly contemplated that the compositions and methods of the instant disclosure can be applied to a number of model systems, to enable assessment of real-time transcriptome monitoring of living cells in their native environment across a time course, optionally in response to administration of agents including, e.g., lead drug compounds, screening compounds, etc. Such real-time/time course transcriptome information is contemplated to aid identification of drug impact upon oncogenesis, cell growth, toxicity and/or drug efficacy for any number of other uses, to the full extent that such real-time/time course transcriptome information can direct compound/lead agent selection, etc.

It is also expressly contemplated that, in certain embodiments, e.g., where the differences between a pathological transcriptome and a normal transcriptome are known, combinations of drugs (including small molecules, biologics, modified nucleic acids, DNA-targeting CRISPR-Cas systems, RNA-targeting CRISPR-Cas systems, TALENs, zinc finger nucleases) can be measured from a living animal to pair phenotype (and/or behavior) with the resulting transcriptome.

### Expression Vector Promoters

An expression vector, otherwise known as an expression construct, is commonly a plasmid or virus designed for gene expression in cells. The vector is used to introduce a specific gene into a target cell, and can commandeer the cell's mechanism for protein synthesis to produce the protein encoded by the gene. Expression vectors are a basic tool in biotechnology for the production of proteins. The vector is engineered to contain regulatory sequences that act as enhancer and promoter regions and lead to efficient transcription of the gene carried on the expression vector. The promoters for cytomegalovirus (CMV) and SV40 are commonly used in mammalian expression vectors to drive gene expression. Non-viral promoters, such as the elongation factor (EF)-1 promoter, are also known.

CMV Promoter is commonly included in vectors used in genetic engineering work conducted in mammalian cells, as it is a strong promoter that drives constitutive expression of genes under its control. This promoter has been used to express a plethora of eukaryotic gene products and is used for specialty protein production, gene therapy, and DNA-based vaccination, among other applications.

The CMV promoter has the following sequence
(SEQ ID NO: 20):
TAGTAATCAATTACGGGGTCATTAGTTCATAGCCCATATATGGAGTTCCG
CGTTACATAACTTACGGTAAATGGCCCGCCTGGCTGACCGCCCAACGACC
CCCGCCCATTGACGTCAATAATGACGTATGTTCCCATAGTAACGCCAATA
GGGACTTTCCATTGACGTCAATGGGTGGAGTATTTACGGTAAACTGCCCA
CTTGGCAGTACATCAAGTGTATCATATGCCAAGTACGCCCCCTATTGACG
TCAATGACGGTAAATGGCCCGCCTGGCATTATGCCCAGTACATGACCTTA
TGGGACTTTCCTACTTGGCAGTACATCTACGTATTAGTCATCGCTATTAC
CATGGTGATGCGGTTTTGGCAGTACATCAATGGGCGTGGATAGCGGTTTG
ACTCACGGGGATTTCCAAGTCTCCACCCCATTGACGTCAATGGGAGTTTG
TTTTGGCACCAAAATCAACGGGACTTTCCAAAATGTCGTAACAACTCCGC
CCCATTGACGCAAATGGGCGGTAGGCGTGTACGGTGGGAGGTCTATATA
AGCAGAGCTGGTTTAGTGAACCGTCAG
The CAG promoter has the following sequence
(SEQ ID NO: 27):
ACATTGATTATTGACTAGTTATTAATAGTAATCAATTACGGGGTCATTA
GTTCATAGCCCATATTGGAGTTCCGCGTTACATAACTTACGGTAAATGG
CCCGCCTGGCTGACCGCCCAACGACCCCCGCCCCTTGACGTCAATAATG
ACGTATGTTCCCATAGTAACGCCAATAGGGACTTTCCATTGACGTCAAT
GGGTGGAGTATTTACGGTAAACTGCCCACTTGGCAGTACATCAAGTGTA
TCATATGCCAAGTACGCCCCCTATTGACGTCAATGACGGTAAATGGCCC
GCCTGGCATTATGCCCAGTACATGACCTTATGGGACTTTCCTACTTGGC
AGTACATCTACGTATTAGTCATCGCTATTACCATGGTGATGCGGTTGTG
GCAGTACATCAATGGGCGTGGATAGCGGTTTGACTCACGGGGATTTCCA
AGTCTCCACCCCATTGACGTCAATGGGAGTTTGTTTTGGCACCAAAATC
AACGGGACTTTCCAAAATGTCGTAACAACTCCGCCCCATTGACGCAAAT
GGGCGGTAGGCGTGTACGGTGGGAGGTCTATATAAGCAGAGCTCGTTTA
GTGAACCGTCAGATCGCCTGGAGACGCCATCCACGCTGTTTTGACCTCC
ATAGAAGACACCGGGACCGATCCAGCCTCCCCTCGAAGCTTTACATGTG
GTACCGAGCTCGGATCCTGAGAACTTCAGGGTGAGTCTATGGGACCCTT
GATGTTTTCTTTCCCCTTCTTTTCTATGGTTAAGTTCATGTCATAGGAA
GGGGAGAAGTAACAGGGTACACATATTGACCAAATCAGGGTAATTTTGC
ATTTGTAATTTTAAAAAATGCTTTCTTCTTTTAATATACTTTTTTGTTT
ATCTTATTTCTAATACTTTCCCTAATCTCTTTCTTTCAGGGCAATAATG
ATACAATGTATCATGCCTCTTTGCACCATTCTAAAGAATAACAGTGATA
ATTTCTGGGTTAAGGCAATAGCAATATTTCTGCATATAAATATTTCTGC
ATATAAATTGTAACTGATGTAAGAGGTTTCATATTGCTAATAGCAGCTA
CAATCCAGCTACCATTCTGCTTTTATTTTATGGTTGGGATAAGGCTGGA
TTATTCTGAGTCCAAGCTAGGCCCTTTTGCTAATCATGTTCATACCTCT
TATCTTCCTCCCACAGCTCCTGGGCAACGTGCTGGTCTGTGTGCTGGCC
CATCACTTTGGC

SV40 Promoter (Simian Virus 40 promoter) contains the SV40 enhancer promoter region and origin of replication (part no. GA-ori-00009.1) for high-level expression and replication in cell lines expressing the large T antigen (e.g. COS-7 and 293T cells). It does not replicate episomally in the absence of the SV40 large T antigen. The SV40 promoter is weak in B cells, but SV40 exhibits high activity in T24 and HCV29 human bladder urethelium carcinoma cell lines.

Human elongation factor-1 alpha (EF-1 alpha) or EF-1 is a constitutive non-viral promoter of human origin that can be used to drive ectopic gene expression in various in vitro and in vivo contexts. EF-1 alpha is often useful in conditions where other promoters (such as CMV) have diminished activity or have been silenced (as in embryonic stem cells).

### Mammalian Cell Culture

In certain aspects, the instant disclosure describes methods and compositions designed to obtain VLP-encapsulated analyte data (e.g., real-time/time course transcriptome data) from living mammalian cells, optionally in cell culture. Mammalian cell culture is used widely in academic, medical and industrial settings. It has provided a means to study the physiology and biochemistry of the cell, and developments in the fields of cell and molecular biology have required the use of reproducible model systems, which cultured cell lines are especially capable of providing. For medical use, cell culture provides test systems to assess the efficacy and toxicology of potential new drugs. Large-scale mammalian cell culture has allowed production of biologically active proteins, initially production of vaccines and then recombinant proteins and monoclonal antibodies; meanwhile, recent innovative uses of cell culture include tissue engineering, as a means of generating tissue substitutes.

Mammalian cells can be isolated from tissues for ex vivo culture in several ways. Cells can be easily purified from blood. However, only the white cells are capable of growth in culture. Cells can be isolated from solid tissues by digesting the extracellular matrix using enzymes such as collagenase, trypsin, or pronase, before agitating the tissue to release the cells into suspension. Alternatively, pieces of tissue can be placed in growth media, and the cells that grow out are available for culture. This method is known as explant culture. Cells that are cultured directly from a subject are known as primary cells. With the exception of some derived from tumors, most primary cell cultures have limited lifespan (Voight et al. Journal of Molecular and Cellular Cardiology. 86: 187-98). An established or immortalized cell line has acquired the ability to proliferate indefinitely either through random mutation or deliberate modification, such as artificial expression of the telomerase gene. Numerous cell lines are well established as representative of particular cell types. Examples of commonly used mammalian cell lines include HEK293T cells, VERO, BHK, HeLa, CV1 (including Cos), MDCK, 293, 3T3, myeloma cell lines (e.g., NSO, NS 1), PC12, WI38 cells, and Chinese hamster ovary (CHO) cells, among many other examples (Langdon et al. Molecular Biomethods Handbook. 861-873).

### Mammalian Cell Transfection Methods

Mammalian cell transfection is a technique commonly used to express exogenous DNA or RNA in a host cell line. There are many different methods available for transfecting mammalian cells, depending upon the cell line characteristics, desired effect, and downstream applications. These methods can be broadly divided into two categories: those used to generate transient transfection, and those used to generate stable transfectants. Transient transfection methods include, but are not limited to, liposome-mediated transfection, non-liposomal transfection agents (lipids and polymers), dendrimer-based transfection, and electroporation. Stable transfection methods include, but are not limited to microinjection, and virus-mediated gene delivery.

In certain aspects of the instant disclosure, stable transfection methods are used, e.g., to achieve integration of exogenous, viral/VLP-forming genes into a mammalian cell genome. Such stable transfection approaches tend to rely upon homologous recombination to achieve directed integration of exogenous nucleic acid sequences, and are well known in the art.

Certain aspects of the instant disclosure describe methods and compositions designed to achieve delivery of exogenous viral genes to mammalian cells. Viral vectors, such as bacteriophages, retrovirus, adenovirus (types 2 and 5), adeno-associated virus, herpes virus, pox virus, human foamy virus (HFV), and lentivirus have been used for gene transfection. Viral vector genomes can be modified by deleting some areas of their genomes so that their replication becomes altered, rendering such viruses safer than native forms. However, viral delivery systems have some problems, including: the marked immunogenicity of viruses, which can cause induction of the inflammatory system, potentially leading to degeneration of transducted tissue; and toxin production, including mortality, the insertional mutagenesis; and their limitation in transgenic capacity size. During the past few years some viral vectors with specific receptors have been designed that are capable of transferring transgenes to some other specific cells, which are not their natural target cells (retargeting) (Nayerossadat et al. Adv Biomed Res. 1: 27).

### Sequencing Methods

Some of the methods and compositions provided herein employ methods of sequencing nucleic acids. A number of DNA sequencing techniques are known in the art, including fluorescence-based sequencing methodologies (See, e.g., Birren et al, Genome Analysis Analyzing DNA, 1, Cold Spring Harbor, N.Y., which is incorporated herein by reference in its entirety). In some embodiments, automated sequencing techniques understood in that art are utilized. In some embodiments, parallel sequencing of partitioned amplicons can be utilized (PCT Publication No WO2006084132, which is incorporated herein by reference in its entirety). In some embodiments, DNA sequencing is achieved by parallel oligonucleotide extension (See, e.g., U.S. Pat. Nos. 5,750,341; 6,306,597, which are incorporated herein by reference in their entireties). Additional examples of sequencing techniques include the Church polony technology (Mitra et al, 2003, Analytical Biochemistry 320, 55-65; Shendure et al, 2005 Science 309, 1728-1732; U.S. Pat. Nos. 6,432,360, 6,485,944, 6,511,803, which are incorporated by reference), the 454 picotiter pyrosequencing technology (Margulies et al, 2005 Nature 437, 376-380; US 20050130173, which are incorporated herein by reference in their entireties), the Solexa single base addition technology (Bennett et al, 2005, Pharmacogenomics, 6, 373-382; U.S. Pat. Nos. 6,787,308; 6,833,246, which are incorporated herein by reference in their entireties), the Lynx massively parallel signature sequencing technology (Brenner et al. (2000). Nat. Biotechnol. 18:630-634; U.S. Pat. Nos. 5,695,934; 5,714,330, which are incorporated herein by reference in their entireties), and the Adessi PCR colony technology (Adessi et al. (2000). Nucleic Acid Res. 28, E87; WO 00018957, which are incorporated herein by reference in their entireties).

Next-generation sequencing (NGS) methods can be employed in certain aspects of the instant disclosure to obtain a high volume of sequence information (such as are particularly required to perform deep sequencing VLPs following capture) in a highly efficient and cost effective manner. NGS methods share the common feature of massively parallel, high-throughput strategies, with the goal of lower costs in comparison to older sequencing methods (see, e.g., Voelkerding et al, Clinical Chem., 55: 641-658, 2009; MacLean et al, Nature Rev. Microbiol, 7-287-296; which are incorporated herein by reference in their entireties). NGS methods can be broadly divided into those that typically use template amplification and those that do not. Amplification-utilizing methods include pyrosequencing commercialized by Roche as the 454 technology platforms (e.g., GS 20 and GS FLX), the Solexa platform commercialized by Illumina®, and the Supported Oligonucleotide Ligation and Detection (SOLiD™) platform commercialized by Applied Biosystems®. Non-amplification approaches, also known as single-molecule sequencing, are exemplified by the HeliScope platform commercialized by Helicos Biosciences, SMRT sequencing commercialized by Pacific Biosciences, and emerging platforms marketed by VisiGen and Oxford Nanopore Technologies Ltd.

In pyrosequencing (U.S. Pat. Nos. 6,210,891; 6,258,568, which are incorporated herein by reference in their entireties), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al, Clinical Chem., 55-641-658, 2009; MacLean et al, Nature Rev. Microbiol, 7:287-296; U.S. Pat. Nos. 6,833,246; 7,115,400; 6,969,488, which are incorporated herein by reference in their entireties), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluorophore and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al, Clinical Chem., 55: 641-658, 2009; U.S. Pat. Nos. 5,912,148; and 6,130,073, which are incorporated herein by reference in their entireties) can initially involve fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing is employed (see, e.g., Astier et al, J. Am. Chem. Soc. 2006 Feb. 8; 128(5): 1705-10, which is incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore (or as individual nucleotides pass through the nanopore in the case of exonuclease-based techniques), this causes a change in the magnitude of the current through the nanopore that is distinct for each of the four bases, thereby allowing the sequence of the DNA molecule to be determined.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, which are incorporated herein by reference in their entireties). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is approximately 99.6% for 50 base reads, with approximately 100 Mb generated per run. The read-length is 100 base pairs. The accuracy for homopolymer repeats of 5 repeats in length is approximately 98%. The benefits of ion semiconductor sequencing are rapid sequencing speed and low upfront and operating costs.

### Kits

The instant disclosure also provides kits containing compositions of the instant disclosure, e.g., for use in methods of the present disclosure. Kits of the instant disclosure may include one or more containers comprising a composition (e.g., a nucleic acid encoding for a virus like particle (VLP) producing protein and a nucleic acid encoding for an epitope-tagged viral surface protein, optionally further including an agent that binds the epitope) of this disclosure. In some embodiments, the kits further include instructions for use in accordance with the methods of this disclosure. In some embodiments, these instructions comprise a description of administration/transfection of the composition(s) to mammalian cells, optionally further including instructions for performance of isolation of VLPs and/or sequencing or other analysis of VLP-encapsulated cytosolic components (e.g., RNAs) produced by mammalian cell(s).

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. The container may further comprise a mammalian cell transfection agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Nucleic Acid Sequences

The following nucleic acid sequences have been used in the instant disclosure:

CAG-Gag-GFP (SEQ ID NO: 24):
ACATTGATTATTGACTAGTTATTAATAGTAATCAATTACGGGGTCATTAGTTCAT
AGCCCATATTGGAGTTCCGCGTTACATAACTTACGGTAAATGGCCCGCCTGGCTG
ACCGCCCAACGACCCCCGCCCCTTGACGTCAATAATGACGTATGTTCCCATAGTA
ACGCCAATAGGGACTTTCCATTGACGTCAATGGGTGGAGTATTTACGGTAAACTG
CCCACTTGGCAGTACATCAAGTGTATCATATGCCAAGTACGCCCCCTATTGACGT
CAATGACGGTAAATGGCCCGCCTGGCATTATGCCCAGTACATGACCTTATGGGAC
TTTCCTACTTGGCAGTACATCTACGTATTAGTCATCGCTATTACCATGGTGATGCG
GTTGTGGCAGTACATCAATGGGCGTGGATAGCGGTTTGACTCACGGGGATTTCCA
AGTCTCCACCCCATTGACGTCAATGGGAGTTTGTTTTGGCACCAAAATCAACGGG
ACTTTCCAAAATGTCGTAACAACTCCGCCCCATTGACGCAAATGGGCGGTAGGCG
TGTACGGTGGGAGGTCTATATAAGCAGAGCTCGTTTAGTGAACCGTCAGATCGCC
TGGAGACGCCATCCACGCTGTTTTGACCTCCATAGAAGACACCGGGACCGATCC
AGCCTCCCCTCGAAGCTTTACATGTGGTACCGAGCTCGGATCCTGAGAACTTCAG
GGTGAGTCTATGGGACCCTTGATGTTTTCTTTCCCCTTCTTTTCTATGGTTAAGTTC
ATGTCATAGGAAGGGGAGAAGTAACAGGGTACACATATTGACCAAATCAGGGTA
ATTTTGCATTTGTAATTTTAAAAAATGCTTTCTTCTTTTAATATACTTTTTTGTTTA
TCTTATTTCTAATACTTTCCCTAATCTCTTTCTTTCAGGGCAATAATGATACAATG
TATCATGCCTCTTTGCACCATTCTAAAGAATAACAGTGATAATTTCTGGGTTAAG
GCAATAGCAATATTTCTGCATATAAATATTTCTGCATATAAATTGTAACTGATGT
AAGAGGTTTCATATTGCTAATAGCAGCTACAATCCAGCTACCATTCTGCTTTTATT
TTATGGTTGGGATAAGGCTGGATTATTCTGAGTCCAAGCTAGGCCCTTTTGCTAA
TCATGTTCATACCTCTTATCTTCCTCCCACAGCTCCTGGGCAACGTGCTGGTCTGT
GTGCTGGCCCATCACTTTGGCAAAGAATTCgccaccATGGGCCAGACTGTTACCACT
CCCTTAAGTTTGACCTTAGGTCACTGGAAAGATGTCGAGCGGATCGCTCACAACC
AGTCGGTAGATGTCAAGAAGAGACGTTGGGTTACCTTCTGCTCTGCAGAATGGCC
AACCTTTAACGTCGGATGGCCGCGAGACGGCACCTTTAACCGAGACCTCATCACC
CAGGTTAAGATCAAGGTCTTTTCACCTGGCCCGCATGGACACCCAGACCAGGTCC
CCTACATCGTGACCTGGGAAGCCTTGGCTTTTGACCCCCCTCCCTGGGTCAAGCC
CTTTGTACACCCTAAGCCTCCGCCTCCTCTTCCTCCATCCGCCCCGTCTCTCCCCC
TTGAACCTCCTCGTTCGACCCCGCCTCGATCCTCCCTTTATCCAGCCCTCACTCCT
TCTCTAGGCGCCAAACCTAAACCTCAAGTTCTTTCTGACAGTGGGGGGCCGCTCA
TCGACCTACTTACAGAAGACCCCCCGCCTTATAGGGACCCAAGACCACCCCCTTC
CGACAGGGACGGAAATGGTGGAGAAGCGACCCCTGCGGGAGAGGCACCGGACC
CCTCCCCAATGGCATCTCGCCTACGTGGGAGACGGGAGCCCCCTGTGGCCGACTC
CACTACCTCGCAGGCATTCCCCCTCCGCGCAGGAGGAAACGGACAGCTTCAATA
CTGGCCGTTCTCCTCTTCTGACCTTTACAACTGGAAAAATAATAACCCTTCTTTTT
CTGAAGATCCAGGTAAACTGACAGCTCTGATCGAGTCTGTCCTCATCACCCATCA
GCCCACCTGGGACGACTGTCAGCAGCTGTTGGGGACTCTGCTGACCGGAGAAGA
AAAACAACGGGTGCTCTTAGAGGCTAGAAAGGCGGTGCGGGGCGATGATGGGCG
CCCCACTCAACTGCCCAATGAAGTCGATGCCGCTTTTCCCCTCGAGCGCCCAGAC
TGGGATTACACCACCCAGGCAGGTAGGAACCACCTAGTCCACTATCGCCAGTTG
CTCCTAGCGGGTCTCCAAAACGCGGGCAGAAGCCCCACCAATTTGGCCAAGGTA
AAAGGAATAACACAAGGGCCCAATGAGTCTCCCTCGGCCTTCCTAGAGAGACTT
AAGGAAGCCTATCGCAGGTACACTCCTTATGACCCTGAGGACCCAGGGCAAGAA
ACTAATGTGTCTATGTCTTTCATTTGGCAGTCTGCCCCAGACATTGGGAGAAAGT
TAGAGAGGTTAGAAGATTTAAAAAACAAGACGCTTGGAGATTTGGTTAGAGAGG
CAGAAAAGATCTTTAATAAACGAGAAACCCCGGAAGAAAGAGAGGAACGTATC
AGGAGAGAAACAGAGGAAAAAGAAGAACGCCGTAGGACAGAGGATGAGCAGA
AAGAGAAAGAAAGAGATCGTAGGAGACATAGAGAGATGAGCAAGCTATTGGCC
ACTGTCGTTAGTGGACAGAAACAGGATAGACAGGGAGGAGAACGAAGGAGGTC
CCAACTCGATCGCGACCAGTGTGCCTACTGCAAAGAAAAGGGGCACTGGGCTAA
AGATTGTCCCAAGAAACCACGAGGACCTCGGGGACCAAGACCGCAGGGATCCGG
CGCAACAAACTTCTCTCTGCTGAAACAAGCCGGAGATGTCGAAGAGAATCCTGG
ACCGATGGAGAGCGACGAGAGCGGCCTGCCCGCCATGAAGATCGAGTGCCGCAT
CACCGGCACCCTGAACGGCGTGGAGTTCGAGCTGGTGGGCGGCGGAGAGGGCAC
CCCCGAGCAGGGCCGCATGACCAACAAGATGAAGAGCACCAAAGGCGCCCTGA
CCTTCAGCCCCTACCTGCTGAGCCACGTGATGGGCTACGGCTTCTACCACTTCGG
CACCTACCCCAGCGGCTACGAGAACCCCTTCCTGCACGCCATCAACAACGGCGG
CTACACCAACACCCGCATCGAGAAGTACGAGGACGGCGGCGTGCTGCACGTGAG
CTTCAGCTACCGCTACGAGGCCGGCCGCGTGATCGGCGACTTCAAGGTGGTGGG
CACCGGCTTCCCCGAGGACAGCGTGATCTTCACCGACAAGATCATCCGCAGCAA
CGCCACCGTGGAGCACCTGCACCCCATGGGCGATAACGTGCTGGTGGGCAGCTT
CGCCCGCACCTTCAGCCTGCGCGACGGCGGCTACTACAGCTTCGTGGTGGACAGC
CACATGCACTTCAAGAGCGCCATCCACCCCAGCATCCTGCAGAACGGGGGCCCC
ATGTTCGCCTTCCGCCGCGTGGAGGAGCTGCACAGCAACACCGAGCTGGGCATC
GTGGAGTACCAGCACGCCTTCAAGACCCCGGATGCAGATGCCGGTGAAGAA
CAG-FLAG-VSVG (K47A, R354A)-mCherry (SEQ ID NO: 25):
ACATTGATTATTGACTAGTTATTAATAGTAATCAATTACGGGGTCATTAGTTCAT
AGCCCATATTGGAGTTCCGCGTTACATAACTTACGGTAAATGGCCCGCCTGGCTG
ACCGCCCAACGACCCCCGCCCCTTGACGTCAATAATGACGTATGTTCCCATAGTA
ACGCCAATAGGGACTTTCCATTGACGTCAATGGGTGGAGTATTTACGGTAAACTG
CCCACTTGGCAGTACATCAAGTGTATCATATGCCAAGTACGCCCCCTATTGACGT
CAATGACGGTAAATGGCCCGCCTGGCATTATGCCCAGTACATGACCTTATGGGAC
TTTCCTACTTGGCAGTACATCTACGTATTAGTCATCGCTATTACCATGGTGATGCG
GTTGTGGCAGTACATCAATGGGCGTGGATAGCGGTTTGACTCACGGGGATTTCCA
AGTCTCCACCCCATTGACGTCAATGGGAGTTTGTTTTGGCACCAAAATCAACGGG
ACTTTCCAAAATGTCGTAACAACTCCGCCCCATTGACGCAAATGGGCGGTAGGCG
TGTACGGTGGGAGGTCTATATAAGCAGAGCTCGTTTAGTGAACCGTCAGATCGCC
TGGAGACGCCATCCACGCTGTTTTGACCTCCATAGAAGACACCGGGACCGATCC
AGCCTCCCCTCGAAGCTTTACATGTGGTACCGAGCTCGGATCCTGAGAACTTCAG
GGTGAGTCTATGGGACCCTTGATGTTTTCTTTCCCCTTCTTTTCTATGGTTAAGTTC
ATGTCATAGGAAGGGGAGAAGTAACAGGGTACACATATTGACCAAATCAGGGTA
ATTTTGCATTTGTAATTTTAAAAAATGCTTTCTTCTTTTAATATACTTTTTTGTTTA
TCTTATTTCTAATACTTTCCCTAATCTCTTTCTTTCAGGGCAATAATGATACAATG
TATCATGCCTCTTTGCACCATTCTAAAGAATAACAGTGATAATTTCTGGGTTAAG
GCAATAGCAATATTTCTGCATATAAATATTTCTGCATATAAATTGTAACTGATGT
AAGAGGTTTCATATTGCTAATAGCAGCTACAATCCAGCTACCATTCTGCTTTTATT
TTATGGTTGGGATAAGGCTGGATTATTCTGAGTCCAAGCTAGGCCCTTTTGCTAA
TCATGTTCATACCTCTTATCTTCCTCCCACAGCTCCTGGGCAACGTGCTGGTCTGT
GTGCTGGCCCATCACTTTGGCAAAGAATTCgccaccATGAAGTGCCTTTTGTACTTA
GCCTTTTTATTCATTGGGGTGAATTGCAAGTTCACCATAGTTTTTCCATCCGGAGG
AGATTACAAGGATGACGACGATAAGGGCGGAAGCTTGGGACACAACCAAAAAG
GAAACTGGAAAAATGTTCCTTCTAATTACCATTATTGCCCGTCAAGCTCAGATTT
AAATTGGCATAATGACTTAATAGGCACAGCCTTACAAGTCAAAATGCCCGCGAG
TCACAAGGCTATTCAAGCAGACGGTTGGATGTGTCATGCTTCCAAATGGGTCACT
ACTTGTGATTTCCGCTGGTATGGACCGAAGTATATAACACATTCCATCCGATCCT
TCACTCCATCTGTAGAACAATGCAAGGAAAGCATTGAACAAACGAAACAAGGAA
CTTGGCTGAATCCAGGCTTCCCTCCTCAAAGTTGTGGATATGCAACTGTGACGGA
TGCCGAAGCAGTGATTGTCCAGGTGACTCCTCACCATGTGCTGGTTGATGAATAC
ACAGGAGAATGGGTTGATTCACAGTTCATCAACGGAAAATGCAGCAATTACATA
TGCCCCACTGTCCATAACTCTACAACCTGGCATTCTGACTATAAGGTCAAAGGGC
TATGTGATTCTAACCTCATTTCCATGGACATCACCTTCTTCTCAGAGGACGGAGA
GCTATCATCCCTGGGAAAGGAGGGCACAGGGTTCAGAAGTAACTACTTTGCTTAT
GAAACTGGAGGCAAGGCCTGCAAAATGCAATACTGCAAGCATTGGGGAGTCAGA
CTCCCATCAGGTGTCTGGTTCGAGATGGCTGATAAGGATCTCTTTGCTGCAGCCA
GATTCCCTGAATGCCCAGAAGGGTCAAGTATCTCTGCTCCATCTCAGACCTCAGT
GGATGTAAGTCTAATTCAGGACGTTGAGAGGATCTTGGATTATTCCCTCTGCCAA
GAAACCTGGAGCAAAATCAGAGCGGGTCTTCCAATCTCTCCAGTGGATCTCAGCT
ATCTTGCTCCTAAAAACCCAGGAACCGGTCCTGCTTTCACCATAATCAATGGTAC
CCTAAAATACTTTGAGACCAGATACATCAGAGTCGATATTGCTGCTCCAATCCTC
TCAAGAATGGTCGGAATGATCAGTGGAACTACCACAGAAGCGGAACTGTGGGAT
GACTGGGCACCATATGAAGACGTGGAAATTGGACCCAATGGAGTTCTGAGGACC
AGTTCAGGATATAAGTTTCCTTTATACATGATTGGACATGGTATGTTGGACTCCG
ATCTTCATCTTAGCTCAAAGGCTCAGGTGTTCGAACATCCTCACATTCAAGACGC
TGCTTCGCAACTTCCTGATGATGAGAGTTTATTTTTTGGTGATACTGGGCTATCCA
AAAATCCAATCGAGCTTGTAGAAGGTTGGTTCAGTAGTTGGAAAAGCTCTATTGC
CTCTTTTTTCTTTATCATAGGGTTAATCATTGGACTATTCTTGGTTCTCCGAGTTGG
TATCCATCTTTGCATTAAATTAAAGCACACCAAGAAAAGACAGATTTATACAGAC
ATAGAGATGAACCGACTTGGAAAGGAATTCGGATCCGGCGCAACAAACTTCTCT
CTGCTGAAACAAGCCGGAGATGTCGAAGAGAATCCTGGACCGatgGTGTCCAAGG
GCGAGGAAGATAACATGGCCATCATCAAGGAGTTCATGAGGTTTAAGGTCCACA
TGGAGGGTTCAGTCAATGGCCACGAGTTCGAGATTGAAGGCGAGGGCGAGGGCC
GCCCCTACGAAGGGACACAGACGGCGAAATTGAAGGTGACCAAAGGCGGGCCA
TTGCCCTTCGCATGGGACATCTTGTCCCCTCAGTTTATGTATGGCAGCAAGGCCT
ATGTTAAGCACCCCGCTGATATCCCGGACTACTTGAAGCTGTCCTTTCCAGAGGG
GTTTAAATGGGAGCGCGTTATGAATTTCGAAGACGGAGGAGTGGTTACGGTGAC
GCAGGACTCATCCCTGCAGGACGGAGAATTTATATATAAGGTTAAGTTGAGAGG
CACAAACTTCCCAAGCGACGGCCCTGTGATGCAGAAGAAAACAATGGGGTGGGA
AGCTTCCAGCGAGCGCATGTACCCCGAAGATGGCGCCCTCAAGGGCGAGATAAA
GCAAAGGCTGAAACTTAAGGACGGCGGTCATTACGACGCGGAGGTCAAGACAAC
TTACAAGGCTAAAAAACCCGTTCAGTTGCCTGGGGCTTACAATGTTAATATCAAACTTGAC
ATCACAAGCCACAATGAAGACTATACGATCGTGGAGCAGTATGAACGAGCGGAAGGCAGGC
ACTCAACGGGGGGGATGGACGAGCTTTACAAG
CAG-HA-VSVG (K47A, R354A)-mCherry (SEQ ID NO: 26):
ACATTGATTATTGACTAGTTATTAATAGTAATCAATTACGGGGTCATTAGTTCAT
AGCCCATATTGGAGTTCCGCGTTACATAACTTACGGTAAATGGCCCGCCTGGCTG
ACCGCCCAACGACCCCCGCCCCTTGACGTCAATAATGACGTATGTTCCCATAGTA
ACGCCAATAGGGACTTTCCATTGACGTCAATGGGTGGAGTATTTACGGTAAACTG
CCCACTTGGCAGTACATCAAGTGTATCATATGCCAAGTACGCCCCCTATTGACGT
CAATGACGGTAAATGGCCCGCCTGGCATTATGCCCAGTACATGACCTTATGGGAC
TTTCCTACTTGGCAGTACATCTACGTATTAGTCATCGCTATTACCATGGTGATGCG
GTTGTGGCAGTACATCAATGGGCGTGGATAGCGGTTTGACTCACGGGGATTTCCA
AGTCTCCACCCCATTGACGTCAATGGGAGTTTGTTTTGGCACCAAAATCAACGGG
ACTTTCCAAAATGTCGTAACAACTCCGCCCCATTGACGCAAATGGGCGGTAGGCG
TGTACGGTGGGAGGTCTATATAAGCAGAGCTCGTTTAGTGAACCGTCAGATCGCC
TGGAGACGCCATCCACGCTGTTTTGACCTCCATAGAAGACACCGGGACCGATCC
AGCCTCCCCTCGAAGCTTTACATGTGGTACCGAGCTCGGATCCTGAGAACTTCAG
GGTGAGTCTATGGGACCCTTGATGTTTTCTTTCCCCTTCTTTTCTATGGTTAAGTTC
ATGTCATAGGAAGGGGAGAAGTAACAGGGTACACATATTGACCAAATCAGGGTA
ATTTTGCATTTGTAATTTTAAAAAATGCTTTCTTCTTTTAATATACTTTTTTGTTTA
TCTTATTTCTAATACTTTCCCTAATCTCTTTCTTTCAGGGCAATAATGATACAATG
TATCATGCCTCTTTGCACCATTCTAAAGAATAACAGTGATAATTTCTGGGTTAAG
GCAATAGCAATATTTCTGCATATAAATATTTCTGCATATAAATTGTAACTGATGT
AAGAGGTTTCATATTGCTAATAGCAGCTACAATCCAGCTACCATTCTGCTTTTATT
TTATGGTTGGGATAAGGCTGGATTATTCTGAGTCCAAGCTAGGCCCTTTTGCTAA
TCATGTTCATACCTCTTATCTTCCTCCCACAGCTCCTGGGCAACGTGCTGGTCTGT
GTGCTGGCCCATCACTTTGGCAAAGAATTCgccaccATGAAGTGCCTTTTGTACTTA
GCCTTTTTATTCATTGGGGTGAATTGCAAGTTCACCATAGTTTTTCCATCCGGAGG
ATACCCATACGATGTTCCAGATTACGCTGGCGGAAGCTTGGGACACAACCAAAA
AGGAAACTGGAAAAATGTTCCTTCTAATTACCATTATTGCCCGTCAAGCTCAGAT
TTAAATTGGCATAATGACTTAATAGGCACAGCCTTACAAGTCAAAATGCCCGCGA
GTCACAAGGCTATTCAAGCAGACGGTTGGATGTGTCATGCTTCCAAATGGGTCAC
TACTTGTGATTTCCGCTGGTATGGACCGAAGTATATAACACATTCCATCCGATCC
TTCACTCCATCTGTAGAACAATGCAAGGAAAGCATTGAACAAACGAAACAAGGA
ACTTGGCTGAATCCAGGCTTCCCTCCTCAAAGTTGTGGATATGCAACTGTGACGG
ATGCCGAAGCAGTGATTGTCCAGGTGACTCCTCACCATGTGCTGGTTGATGAATA
CACAGGAGAATGGGTTGATTCACAGTTCATCAACGGAAAATGCAGCAATTACAT
ATGCCCCACTGTCCATAACTCTACAACCTGGCATTCTGACTATAAGGTCAAAGGG
CTATGTGATTCTAACCTCATTTCCATGGACATCACCTTCTTCTCAGAGGACGGAG
AGCTATCATCCCTGGGAAAGGAGGGCACAGGGTTCAGAAGTAACTACTTTGCTT
ATGAAACTGGAGGCAAGGCCTGCAAAATGCAATACTGCAAGCATTGGGGAGTCA
GACTCCCATCAGGTGTCTGGTTCGAGATGGCTGATAAGGATCTCTTTGCTGCAGC
CAGATTCCCTGAATGCCCAGAAGGGTCAAGTATCTCTGCTCCATCTCAGACCTCA
GTGGATGTAAGTCTAATTCAGGACGTTGAGAGGATCTTGGATTATTCCCTCTGCC
AAGAAACCTGGAGCAAAATCAGAGCGGGTCTTCCAATCTCTCCAGTGGATCTCA
GCTATCTTGCTCCTAAAAACCCAGGAACCGGTCCTGCTTTCACCATAATCAATGG
TACCCTAAAATACTTTGAGACCAGATACATCAGAGTCGATATTGCTGCTCCAATC
CTCTCAAGAATGGTCGGAATGATCAGTGGAACTACCACAGAAGCGGAACTGTGG
GATGACTGGGCACCATATGAAGACGTGGAAATTGGACCCAATGGAGTTCTGAGG
ACCAGTTCAGGATATAAGTTTCCTTTATACATGATTGGACATGGTATGTTGGACT
CCGATCTTCATCTTAGCTCAAAGGCTCAGGTGTTCGAACATCCTCACATTCAAGA
CGCTGCTTCGCAACTTCCTGATGATGAGAGTTTATTTTTTGGTGATACTGGGCTAT
CCAAAAATCCAATCGAGCTTGTAGAAGGTTGGTTCAGTAGTTGGAAAAGCTCTAT
TGCCTCTTTTTTCTTTATCATAGGGTTAATCATTGGACTATTCTTGGTTCTCCGAGT
TGGTATCCATCTTTGCATTAAATTAAAGCACACCAAGAAAAGACAGATTTATACA
GACATAGAGATGAACCGACTTGGAAAGGAATTCGGATCCGGCGCAACAAACTTC
TCTCTGCTGAAACAAGCCGGAGATGTCGAAGAGAATCCTGGACCGatgGTGTCCA
AGGGCGAGGAAGATAACATGGCCATCATCAAGGAGTTCATGAGGTTTAAGGTCC
ACATGGAGGGTTCAGTCAATGGCCACGAGTTCGAGATTGAAGGCGAGGGCGAGG
GCCGCCCCTACGAAGGGACACAGACGGCGAAATTGAAGGTGACCAAAGGCGGG
CCATTGCCCTTCGCATGGGACATCTTGTCCCCTCAGTTTATGTATGGCAGCAAGG
CCTATGTTAAGCACCCCGCTGATATCCCGGACTACTTGAAGCTGTCCTTTCCAGA
GGGGTTTAAATGGGAGCGCGTTATGAATTTCGAAGACGGAGGAGTGGTTACGGT
GACGCAGGACTCATCCCTGCAGGACGGAGAATTTATATATAAGGTTAAGTTGAG
AGGCACAAACTTCCCAAGCGACGGCCCTGTGATGCAGAAGAAAACAATGGGGTG
GGAAGCTTCCAGCGAGCGCATGTACCCCGAAGATGGCGCCCTCAAGGGCGAGAT
AAAGCAAAGGCTGAAACTTAAGGACGGCGGTCATTACGACGCGGAGGTCAAGAC
AACTTACAAGGCTAAAAAACCCGTTCAGTTGCCTGGGGCTTACAATGTTAATATCAAACTT
GACATCACAAGCCACAATGAAGACTATACGATCGTGGAGCAGTATGAACGAGCGGAAGGCA
GGCACTCAACGGGGGGGATGGACGAGCTTTACAAG

Transfections were carried out using Lipofectamine® 2000, following manufacturer's instructions. Transductions were carried out by transducing cells with lentivirus carrying transgenes, and then selection was performed either using antibiotics (such as puromycin) or by flow cytometry (if using a fluorescent protein such as GFP). VLPs were harvested from media and then purified by centrifuging at 2000 rcf for 10 minutes at 4° C., then filtering the supernatant with a 0.45 μm cellulose acetate filter. The VLPs were optionally further concentrated via ultracentrifugation (using a 20% sucrose cushion) or using a 100 k MWCF (such as an amicon filter) and centrifuging for 4000 rcf for 30 minutes at 4° C. The concentrated VLPs were optionally further purified via immunoprecipitation with appropriate beads (such as Anti-FLAG M2 magnetic beads) and following manufacturer's instructions. RNAseq was carried out using SMART-Seq.

### Example 2: Induction of Mammalian Cell Production of Epitope-Tagged VLPs

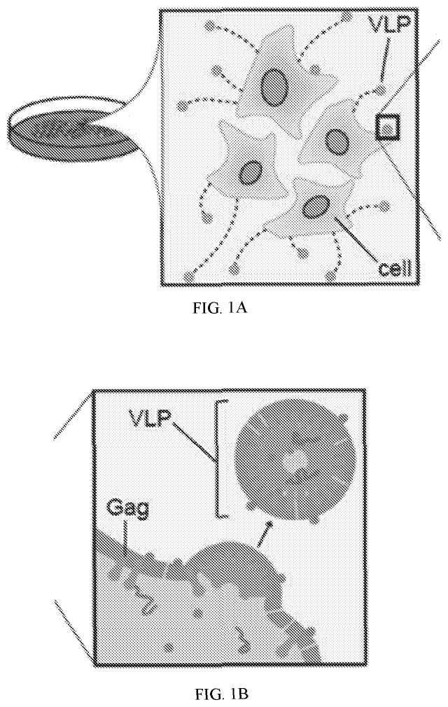

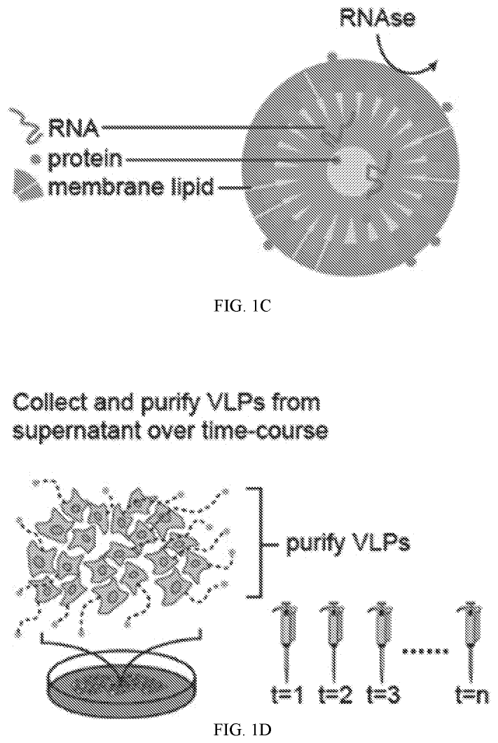

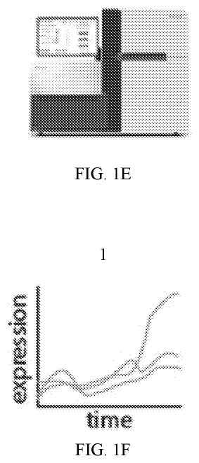

In retroviruses, VLPs have been described as generated by endogenous or ectopic expression of gag or gag-like proteins. Recognizing that VLPs encapsulate cytosolic components of their originating cells, including proteins, lipids, metabolites, small molecules, RNA and/or DNA (see FIGS. 1A and 1B), it was first examined whether VLP formation could be induced in mammalian cells. Once induced, it was contemplated herein that VLPs could then be specifically isolated as a means of measuring transcriptome expression levels in living cells in real time/across a time course. For purification of VLPs, it was examined whether VLPs could be tagged (e.g., via introduction of epitope tags such as HA, FLAG, etc.), thereby allowing for immunoprecipitation (IP) or other affinity-based methods to isolate VLPs (i.e. via IP of cell line-specific and/or virion-specific tags; FIG. 1D). In addition or as an alternative, it was also contemplated that VLPs could be isolated via centrifugation, concentration via molecular weight cutoff filters, gradients, crowding agents (such as PEG), or a combination of the aforementioned methods. It was contemplated herein that the VLPs produced by a mammalian cell harboring a VLP producing protein could be isolated and used to assess a variety of mammalian cell analytes in real time/across a time course, while maintaining an intact and alive mammalian cell throughout such a monitoring period. Further, it was specifically contemplated that VLPs could be isolated and used to assess RNA as the mammalian cell analyte tracked in real-time/across a time course and at high throughput, via application of Next Generation Sequencing (NGS) technologies to such isolated populations of VLPs (FIG. 1E). It was projected that expression profiling results could be determined over time from the same biological sample(s) via transcriptome-directed assessment of VLPs (FIG. 1F).

### Example 3: Epitope-Tagged VLPs Provided High Quality RNA Libraries

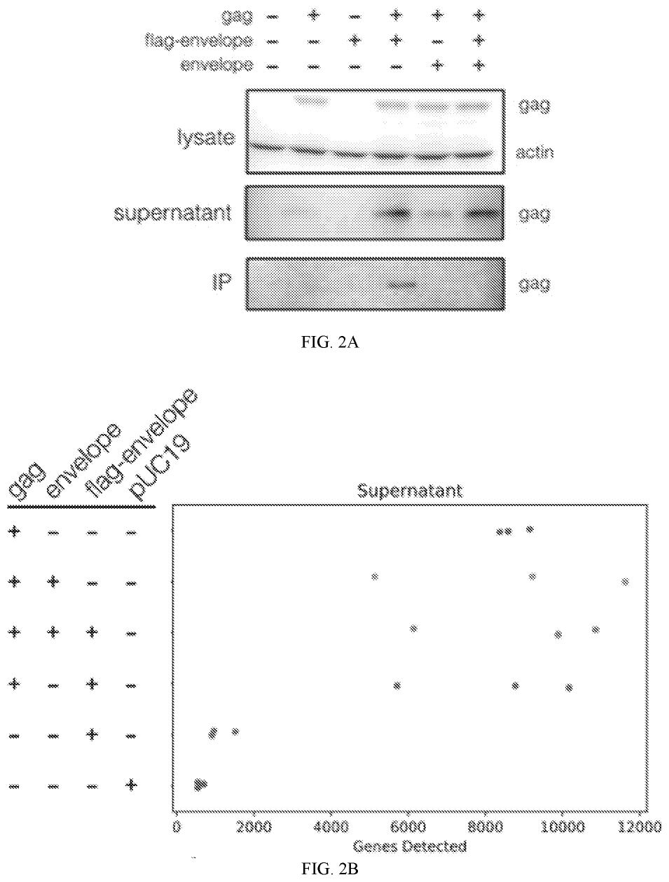

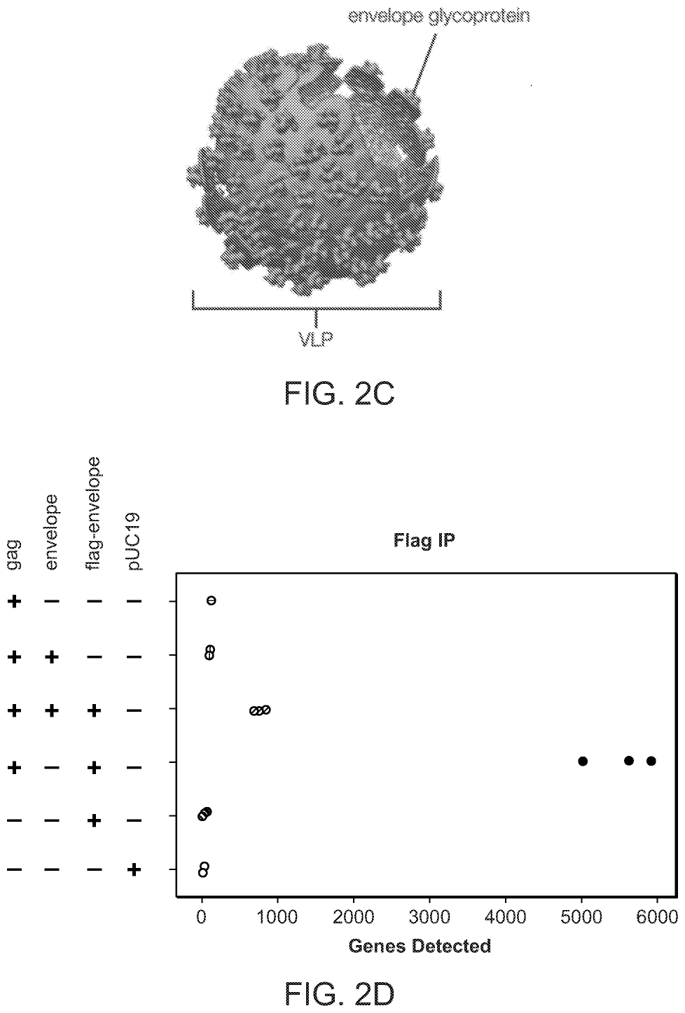

Nucleic acids encoding for a retroviral gag protein (specifically, MLV gag) and a flag epitope-tagged envelope protein (here, flag-VSV-g) were introduced into mammalian 293T cells. Immunoprecipitation was performed upon the flag epitope tag, and western blot results confirmed that gag protein could be specifically and cleanly isolated via epitope-mediated IP from supernatant, which confirmed that VLPs were successfully isolated (FIG. 2A). VLP samples were then sequenced for RNA content, and dot plot data of supernatants obtained from conditions where MLV gag was transfected demonstrated that the instant approach generated high quality RNA libraries, as measured by genes detected (which confirmed both the depth and diversity of the VLP-derived RNA libraries; FIG. 2B). Background from VLPs lacking flag-labeled envelopes was identified as negligible (FIG. 2D).

### Example 4: Distinct Epitope Tags Readily Distinguished Cell Lines of Origin, Even in Mixed Cell Culture

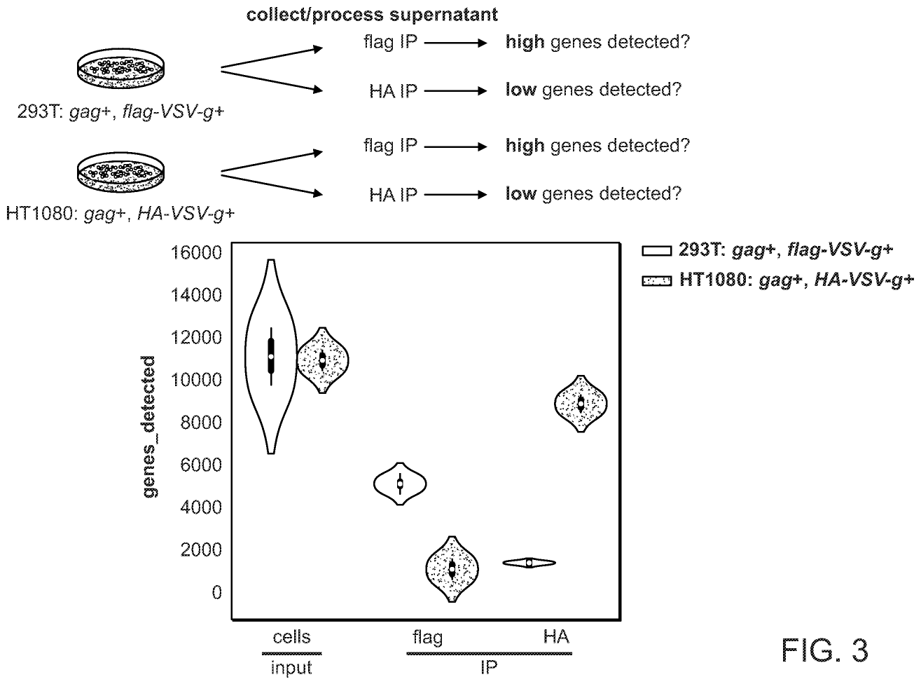

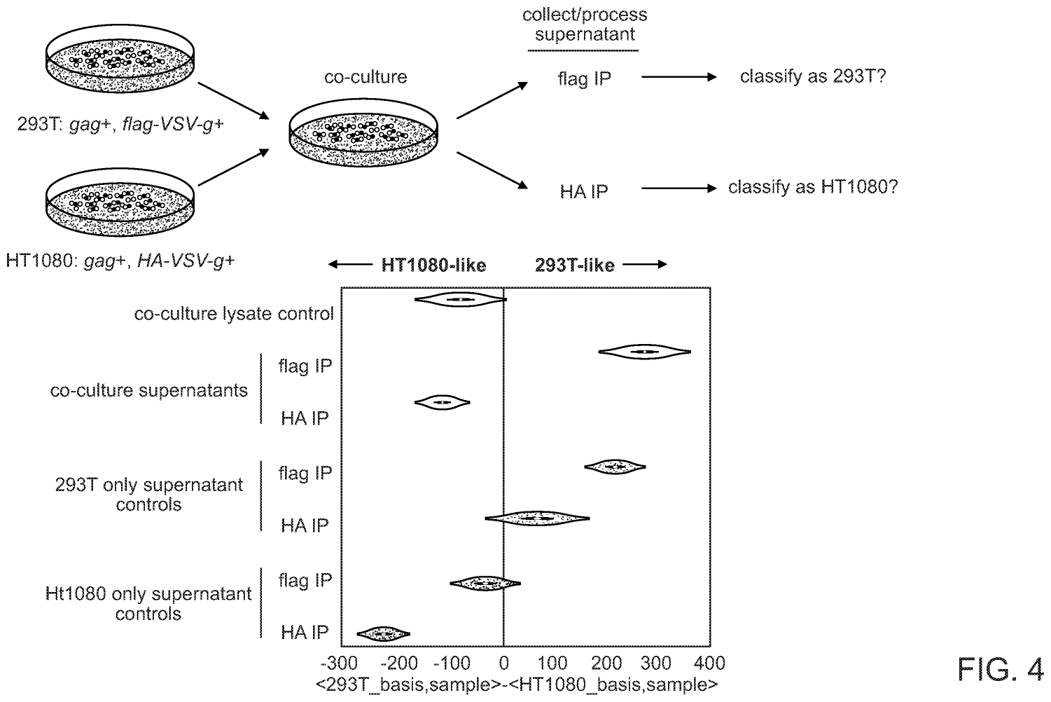

To examine whether different mammalian cell lines of origin could provide real-time/time course analyte information that was independently identifiable as attributable to VLPs generated by respective types of mammalian cell lines, two distinct cell lines (here, 293T cells and HT1080) were administered retroviral gag and either flag epitope-labeled envelope (flag-VSV-G) constructs or HA epitope-labeled envelope constructs (HA-VSV-G). As shown in FIGS. 3A and 3B, populations of VLPs from each cell type could be readily distinguished at the transcriptome/genes detected level, based upon the respective epitope tags used correlating with depth of sequence coverage obtained. Specifically, high quality transcript libraries (quantified by genes detected at sequencing) were generated when each supernatant was put through the matching immunoprecipitation step. Conversely, poor quality transcript (RNAseq) libraries (quantified by genes detected) were generated when each supernatant was put through an incorrect (unmatched) immunoprecipitation (FIG. 3B).

It was next examined if mixed cell populations presenting distinct epitope tags on their VLP envelope proteins (thereby distinguishing their respective VLPs at origin, before mixing) could be identified in co-culture based upon these epitope tags. As shown in FIG. 4, epitope tag-isolated VLPs were readily distinguished as reflecting their cell type of origin (here, 293T or HT1080), even when obtained from a mixed cellular population in culture.

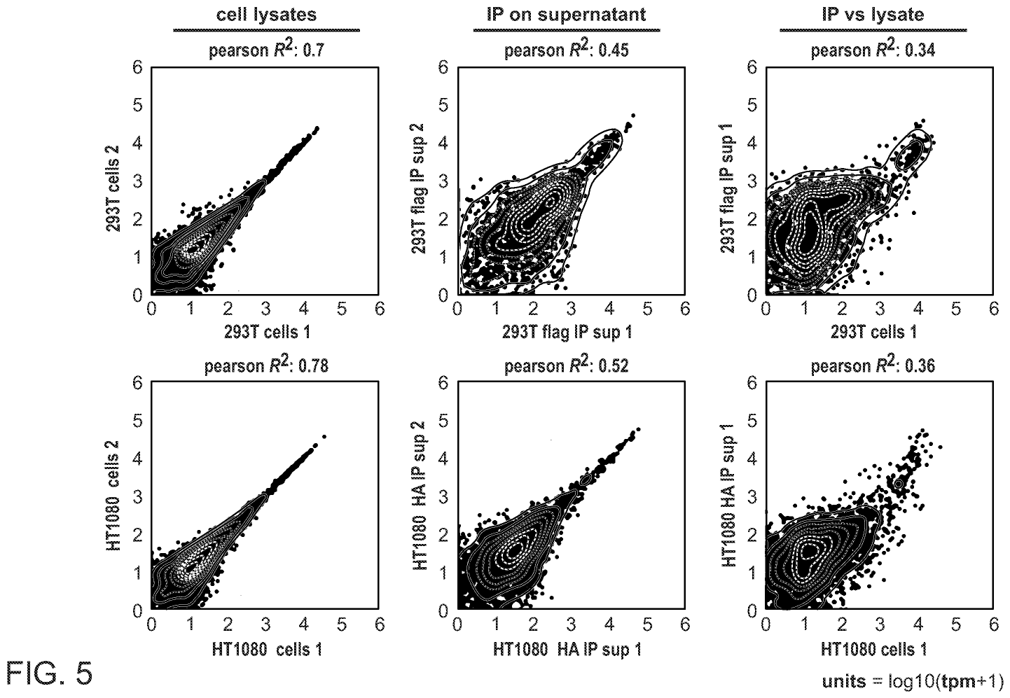

Additional transcript sequencing (RNAseq) data also confirmed that quantitative transcriptional information could be measured from live-cell co-cultures via purification of affinity-tagged VLPs via immunoprecipitation (FIG. 5), as demonstrated by the extensive correlation observed between assays.

### Example 5: Transcriptome Monitoring of Living Cells in Co-Culture and in Model Systems

The compositions and methods of the instant disclosure can be employed to provide significant insight in in vitro screens with complex cell populations, where RNA information is desired from each sub-population of cells. Populations can be independently modified with the constructs, and subsequently pooled together, or specific promoters can be used to specifically label desired populations. A specifically contemplated example of such a system is an in vitro screen performed upon primary cortical neurons, such as E18 rat or mouse cortical neurons. Such cultures contain several cell types, such as excitatory neurons, inhibitory neurons and glia. In certain aspects, to examine the effects of perturbations in such cells caused by small molecules, siRNA/shRNA, CRISPRi/a, gene knockout (via CRISPR or other methods), metabolites, viruses, proteins, peptides, photons (with or without optogenetics), ORFs, prokaryotes, or other eukaryotic cells, optionally during screening, the following compositions and methods are employed. Successful labeling of VLPs from excitatory neurons is performed by using a CamKII promoter to drive expression of both a VLP producing protein, such as MLV Gag, as well as a labeled envelope protein, such as FLAG-VSVG. Meanwhile, successful labeling of VLPs from inhibitory neurons is performed using a mDIx promoter to drive expression of both a VLP producing protein, such as MLV Gag, as well as a labeled envelope protein, such as HA-VSVG. In this example, these subpopulations are simultaneously modified by delivering constructs driven by these cell-type specific promoters via AAV (optionally together with adenovirus), lentivirus, or transfection. Particles are collected by sampling the supernatant, performing a 2000 rcf centrifugation for 10 minutes at 4° C., and then incubating in the appropriate immunoprecipitation or affinity beads and performing the capture and elution per manufacturer's instructions. After the elution of the particles, RNA sequencing (optionally by RNAseq), mass spectrometry, western blot, northern blot, microarray, luminex, droplet based library construction (such as 10× single cell 3′), or other detection method, is performed.

The instant disclosure is also contemplated as providing significant insight in an animal model where cells are modified with the constructs of the disclosure ex vivo and then subsequently implanted. An example of this is a cancer model in mice, where ex vivo modified gag+flag-vsvg+luciferase+HT1080 cells or 293T cells are administered via intraperitoneal injection. Cell invasion and metastasis can be monitored via luciferase detection using standard methods, and this cellular behavior can be coupled with VLP-derived analyte (e.g., transcriptome sequences, e.g., RNAseq) information via blood sampling such as sampling via tail vein. The resulting samples can be processed using affinity capture and then analyte assessment (e.g., RNAseq) can be performed. In this example, in vivo metastasis information can optionally be coupled with analyte assessment (e.g., RNAseq) information, in order to better assess and understand mechanisms of metastasis in vivo.

The compositions and methods of the instant disclosure can also provide significant insight in an animal model where cells are modified with the constructs in vivo. For example, the constructs described herein can be packaged in AAV with a modified capsid that permits blood brain barrier crossing, such as AAV-PHP.B. This AAV can be delivered via intravenous injection, thus modifying neurons directly in vivo. To examine the transcriptomes of different neuronal cell-types in vivo, cerebral spinal fluid can be collected and processed, selecting the exported particles via affinity capture and elution. After the elution of the particles, VLP-captured analyte assessment vie transcriptome detection (e.g., via RNAseq), mass spectrometry, western blot, northern blot, microarray, luminex, droplet based library construction (such as 10× single cell 3′), can be performed, in order to couple freely-behaving animal observations such as development, behavior and/or in vivo perturbations with high-throughput molecular readouts.

A strength of the compositions and methods of the instant disclosure is that the compositions and methods can be used to monitor broad transcriptional information, rather than focus on a few genes. The methods are particularly well suited for cases where cells are implanted/modified in vivo, or are non-dividing in vitro. One particularly interesting expressly contemplated use case is an engineered patient-derived xenografts (PDX) model for glioblastoma multiforme (GBM) in immunodeficient mice. Using an approach of the instant disclosure, transcriptional responses from CSF are gathered after administering different small molecules or biologics possessing potential therapeutic impact. The transcriptional data are used to measure gene expression across all gene ontologies of interest, which include cell cycle regulation, apoptosis, stemness/pluripotency, and/or differentiation. Further, since the instant compositions and methods capture full-length transcriptional information, mutations can be identified and mechanisms of resistance can be deciphered as therapeutic pressure is strengthened in a particular animal model.

Thus, expressly contemplated applications for the compositions and methods of the instant disclosure include in vitro studies of complex cellular populations (such as primary cortical neurons, which have excitatory neurons, inhibitory neurons, glia and other cell types, as described in additional detail above), and assessment of implanted cells (such as cancerous cells) in animal models (in the case of studying implanted cells in animal models, it is contemplated that cells are optionally infected ex vivo before implantation or are infected in vivo/in situ, e.g., via viral or other directed modes of gene delivery in vivo), with VLP-captured analytes (e.g., transcription changes) then monitored as VLP-producing cells invade or proliferate in such animal models, and/or as agents (e.g., candidate drugs) are administered to the animal model.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A mammalian cell comprising a virus like particle (VLP) producing protein and an epitope-tagged viral surface protein.

2. A composition selected from the group consisting of:
A mammalian cell comprising an epitope-tagged virus like particle (VLP) producing protein; and
A mammalian cell comprising a virus like particle (VLP) producing protein and a viral surface protein of a virus differing from the virus of the virus like particle (VLP) producing protein.

3. (canceled)

4. The composition of claim 2, wherein the VLP is a non-enveloped virus VLP, optionally wherein the non-enveloped virus comprises an engineered affinity tag, optionally wherein the non-enveloped virus VLP is selected from the group consisting of an Adenoviridae, a Papovaviridae, a Parvoviridae, and an Anelloviridae family virus.

5. The mammalian cell of claim 1, wherein the virus like particle (VLP) producing protein is a retroviral gag protein or a viral gag-like protein, optionally wherein the viral gag protein is selected from the group consisting of a murine leukemia virus (MLV) gag protein, a retrovirus matrix protein, a rhabdovirus matrix (M) protein (optionally VSVM protein), a filovirus viral core protein (optionally an Ebola VP40 viral protein), a Rift Valley Fever virus N protein (optionally RVFV N Protein having GenBank serial number NP049344), a coronavirus M, E and/or NP protein (optionally GenBank serial number NP040838 for NP protein, GenBank serial number NP 040835 for M protein, GenBank serial number CAC39303 for E protein of Avian Infections Bronchitis Virus and GenBank serial number NP828854 for E protein of the SARS virus), a bunyavirus N protein (optionally the bunyavirus N protein of GenBank serial number AAA47114), an influenza M1 protein, a paramyxovirus M protein, an arenavirus Z protein (optionally a Lassa Fever Virus Z protein), an AAV gag-like protein (optionally selected from the group consisting of AAV1 capsid, AAV2 capsid, AAV3 capsid, AAV4 capsid, AAV5 capsid, AAV6 capsid, AAV7 capsid, AAV8 capsid, AAV9 capsid, AAV10 capsid, AAV11 capsid, AAV12 capsid, and AAV13 capsid), and combinations thereof.

6. The mammalian cell of claim 1, wherein the epitope-tagged viral surface protein is a Vesicular Stomatitis Virus (VSV) glycoprotein (VSV-G) or a mutagenized form of VSV-G, optionally wherein the mutagenized form of VSV-G prevents VSV-G-mediated cellular uptake.

7. The mammalian cell of claim 1, wherein the epitope-tagged viral surface protein is an epitope-tagged viral envelope protein, optionally wherein the epitope-tagged viral envelope protein is selected from the group consisting of an epitope-tagged form of any of the following: a Vesicular Stomatitis Virus (VSV) glycoprotein, a retrovirus glycoprotein (optionally a human immunodeficiency virus (HIV) envelope glycoprotein (optionally HIVSF162 envelope glycoprotein of GenBank serial number M65024)), a simian immunodeficiency virus (SIV) envelope glycoprotein (optionally SIVmac239 envelope glycoprotein of GenBank serial number M33262), a simian-human immunodeficiency virus (SHIV) envelope glycoprotein (optionally SHIV-89.6p envelope glycoprotein of GenBank serial number U89134), a feline immunodeficiency virus (FIV) envelope glycoprotein (optionally FIV envelope glycoprotein of GenBank serial number L00607), a feline leukemia virus (FLV) envelope glycoprotein (optionally the FLV envelope glycoprotein of GenBank serial number M12500), a bovine immunodeficiency virus (BIV) envelope glycoprotein (optionally the BIV envelope glycoprotein of GenBank serial number NC001413), a bovine leukemia virus (BLV) envelope glycoprotein (optionally of GenBank serial number AF399703), an equine infectious anemia virus envelope glycoprotein (optionally the equine infectious anemia virus envelope glycoprotein of GenBank serial number NC001450), a human T-cell leukemia virus envelope glycoprotein (optionally the human T-cell leukemia virus envelope glycoprotein of GenBank serial number AF0033817), a mouse mammary tumor virus envelope glycoprotein (MMTV), a bunyavirus glycoprotein (optionally a Rift Valley Fever virus (RVFV) glycoprotein (optionally the RVFV envelope glycoprotein of GenBank serial number M11157)), an arenavirus glycoprotein (optionally a Lassa fever virus glycoprotein (optionally of GenBank serial number AF333969))), a filovirus glycoprotein (e.g., an Ebola virus glycoprotein (GenBank serial number NC002549)), a corona virus glycoprotein (optionally of GenBank serial number SARS coronavirus spike protein AAP13567), an influenza virus glycoprotein (optionally of GenBank serial number V01085), a paramyxovirus glycoprotein (optionally of GenBank serial number NC002728 for Nipah virus F and G proteins), a rhabdovirus glycoprotein (optionally of GenBank serial number NP049548)), an alphavirus glycoprotein (optionally of GenBank serial number AAA48370 for Venezuelan equine encephalomyelitis (VEE)), a flavivirus glycoprotein (optionally of GenBank serial number NC001563 for West Nile virus and/or a Hepatitis C Virus glycoprotein), a Herpes Virus glycoprotein (optionally a cytomegalovirus glycoprotein), and combinations thereof.

8. The mammalian cell of claim 1, wherein the epitope-tagged viral surface protein is selected from the group consisting of Coronavirus gpE1, Coronavirus Peplomer Protein E1, Coronavirus Peplomer Protein E2 JHM, Hepatitis Virus (MHV), Glycoprotein E2, LaCrosse Virus Envelope Glycoprotein G1, Simian Sarcoma Virus Glycoprotein 70, Viral Envelope Glycoprotein gp55 (Friend Virus), and Viral Envelope Glycoprotein gPr90 (Murine Leukemia Virus).

9. The mammalian cell of claim 1, wherein the epitope tag is selected from the group consisting of FLAG (DYKDDDDK; SEQ ID NO: 3), 6×His (HHHHHH; SEQ ID NO: 4), HA (YPYDVPDYA; SEQ ID NO: 5), c-myc (EQKLISEEDL; SEQ ID NO: 6), V5 tag (GKPIPNPLLGLDST; SEQ ID NO: 7), AU1 tag (DTYRYI; SEQ ID NO: 8), AU5 tag (TDFYLK; SEQ ID NO: 9), Glu-Glu tag (EYMPME; SEQ ID NO: 10), OLLAS (SGFANELGPRLMGK; SEQ ID NO: 11), T7 tag (MASMTGGQQMG; SEQ ID NO: 12), VSV-G tag (YTDIEMNRLGK; SEQ ID NO: 13), E-Tag (GAPVPYPDPLEPR; SEQ ID NO: 14), S-Tag (KETAAAKFERQHMDS; SEQ ID NO: 15), HSV tag (SQPELAPEDPED; SEQ ID NO: 16), KT3 tag (KPPTPPPEPET; SEQ ID NO: 17), TK15 tag, GST tag, Protein A tag, CD tag, Strep-Tag (WSHPQFEK; SEQ ID NO: 18), MBP tag, CBD tag, Avi tag (CGLNDIFEAQKIEWHE; SEQ ID NO: 19), CBP tag, TAP tag, and SF-TAP tag.

10. The mammalian cell of claim 1, wherein the mammalian cell is infected by a virus, optionally wherein the mammalian cell is infected by AAV (and optionally adenovirus, HPV or other virus), or by a retrovirus, optionally wherein the retrovirus is a lentivirus.

11. The mammalian cell of claim 1, wherein the mammalian cell is a cell in culture.

12. The mammalian cell of claim 1, wherein the mammalian cell is a neuronal cell, optionally a primary cortical neuron, optionally an excitatory neuron or an inhibitory neuron.

13. The mammalian cell of claim 1, wherein the mammalian cell is a cell in vivo.

14. The mammalian cell of claim 1, wherein the VLP producing protein and/or the epitope-tagged viral surface protein are produced by the mammalian cell via a genomically integrated nucleic acid sequence that encodes for the VLP producing protein and/or the epitope-tagged viral surface protein, optionally wherein the nucleic acid sequence that encodes for the VLP producing protein and/or the epitope-tagged viral surface protein is under the control of a mammalian promoter, optionally a CMV promoter, a SV40 promoter and/or a tissue-specific mammalian promoter (optionally a mDIx, CamKII, Syn1, NSE, PDGF and/or Ta1 promoter, optionally a CamKII promoter and/or a mDIx promoter).

15. A method selected from the group consisting of:
A method for obtaining an expression profile of a living cell, the method comprising:
(a) providing a living cell;
(b) introducing a nucleic acid sequence encoding for a VLP producing protein to the living cell, wherein introduction of the nucleic acid sequence encoding for a VLP producing protein is sufficient to induce budding of VLPs from the living cell;
(c) isolating VLPs produced by the living cell via binding of a VLP protein; and
(d) performing RNA sequencing upon the isolated VLPs, thereby obtaining expression profile information for the isolated VLPs, wherein the expression profile information for the isolated VLPs reflects the expression profile of the living cell,
thereby obtaining an expression profile of the living cell;
A method for obtaining an expression profile of a living cell, the method comprising:
(a) providing a living cell;
(b) introducing a first nucleic acid sequence encoding for a VLP producing protein and a second nucleic acid encoding for an epitope-tagged viral surface protein to the living cell, wherein introduction of the first nucleic acid sequence encoding for a VLP producing protein is sufficient to induce budding of VLPs from the living cell;
(c) isolating VLPs produced by the living cell via binding of the epitope-tagged viral surface protein; and
(d) performing RNA sequencing upon the isolated VLPs, thereby obtaining expression profile information for the isolated VLPs, wherein the expression profile information for the isolated VLPs reflects the expression profile of the living cell,
thereby obtaining an expression profile of the living cell;
A method for obtaining a first analyte profile for a first population of living cells and a second analyte profile for a second population of living cells, the method comprising:
(a) providing a first population of living cells;
(b) introducing a first nucleic acid sequence encoding for a VLP producing protein and a second nucleic acid encoding for a first epitope-tagged viral surface protein to the first population of living cells, wherein introduction of the first nucleic acid sequence encoding for a VLP producing protein is sufficient to induce budding of VLPs from the first population of living cells;
(c) providing a second population of living cells;
(d) introducing the first nucleic acid sequence encoding for a VLP producing protein and a second nucleic acid sequence encoding for a second epitope-tagged viral surface protein to the second population of living cells, wherein introduction of the first nucleic acid sequence comprising a nucleic acid sequence encoding for a VLP producing protein is sufficient to induce budding of VLPs from the second population of living cells;
(e) isolating VLPs produced by the first population of living cells via binding of the first epitope-tagged viral surface protein;
(f) obtaining a first analyte profile from the isolated VLPs of the first population of living cells;
(g) isolating VLPs produced by the second population of living cells via binding of the second epitope-tagged viral surface protein; and
(h) obtaining a second analyte profile from the isolated VLPs of the second population of living cells,
thereby obtaining a first analyte profile for a first population of living cells and a second analyte profile for a second population of living cells;
A method for obtaining a first analyte profile for a first population of living cells and a second analyte profile for a second population of living cells, the method comprising:
(a) providing a first population of living cells;
(b) introducing a first nucleic acid sequence encoding for a VLP producing protein to the first population of living cells, wherein introduction of the first nucleic acid sequence encoding for a VLP producing protein is sufficient to induce budding of VLPs from the first population of living cells;
(c) providing a second population of living cells;
(d) introducing a second nucleic acid sequence encoding for a VLP producing protein to the second population of living cells, wherein introduction of the second nucleic acid sequence encoding for a VLP producing protein is sufficient to induce budding of VLPs from the second population of living cells;
(e) isolating VLPs produced by the first population of living cells via binding of a first VLP protein;
(f) obtaining a first analyte profile from the isolated VLPs of the first population of living cells;
(g) isolating VLPs produced by the second population of living cells via binding of a second VLP protein; and
(h) obtaining a second analyte profile from the isolated VLPs of the second population of living cells,
thereby obtaining a first analyte profile for a first population of living cells and a second analyte profile for a second population of living cells;
A method for assessing a test compound for efficacy and/or toxicity in living cells, the method comprising:
(a) providing a population of living cells;
(b) introducing a nucleic acid sequence encoding for a VLP producing protein to the living cells, wherein introduction of the nucleic acid sequence encoding for the VLP producing protein is sufficient to induce budding of VLPs from the living cells;
(c) contacting the living cells with a test compound;
(d) isolating VLPs produced by the living cells via binding of a VLP protein; and
(e) obtaining analyte profile information from the isolated VLPs, wherein the analyte profile information indicates the efficacy and/or toxicity of the test compound,
thereby assessing a test compound for efficacy and/or toxicity in living cells; and
A method for assessing a test compound for efficacy and/or toxicity in living cells, the method comprising:
(a) providing a population of living cells;
(b) introducing a first nucleic acid sequence encoding for a VLP producing protein and a second nucleic acid sequence encoding for an epitope-tagged viral surface protein to the living cells, wherein introduction of the nucleic acid sequence encoding for a VLP producing protein is sufficient to induce budding of VLPs from the living cells;
(c) contacting the living cells with a test compound;
(d) isolating VLPs produced by the living cells via binding of the epitope-tagged viral surface protein; and
(e) obtaining analyte profile information from the isolated VLPs, wherein the analyte profile information indicates the efficacy and/or toxicity of the test compound,
thereby assessing a test compound for efficacy and/or toxicity in living cells.

16. The method for obtaining an expression profile of claim 15, wherein the VLP protein of step (c) is the VLP producing protein, optionally wherein the VLP producing protein is tagged, optionally wherein the tag is an epitope tag.

17. The method of claim 15, wherein:
the VLP is a non-enveloped virus VLP, optionally wherein the non-enveloped virus comprises an engineered affinity tag, optionally wherein the non-enveloped virus VLP is selected from the group consisting of an Adenoviridae, a Papovaviridae, a Parvoviridae, and an Anelloviridae family virus;
the VLP protein of step (c) is a capsid protein of the VLP or an envelope protein of the VLP, optionally wherein the capsid protein of the VLP or the envelope protein of the VLP is tagged, optionally wherein the tag is an epitope tag;
the VLP protein of step (c) is a host cell membrane protein, optionally an affinity-tagged host cell membrane protein; and/or
an antibody is used to bind the VLP protein in step (c), optionally wherein the antibody binds the VLP producing protein, a capsid protein of the VLP, and/or an envelope protein of the VLP.

18-22. (canceled)

23. The method of claim 16, wherein the epitope tag is selected from the group consisting of FLAG (DYKDDDDK; SEQ ID NO: 3), 6×His (HHHHHH; SEQ ID NO: 4), HA (YPYDVPDYA; SEQ ID NO: 5), c-myc (EQKLISEEDL; SEQ ID NO: 6), V5 tag (GKPIPNPLLGLDST; SEQ ID NO: 7), AU1 tag (DTYRYI; SEQ ID NO: 8), AU5 tag (TDFYLK; SEQ ID NO: 9), Glu-Glu tag (EYMPME; SEQ ID NO: 10), OLLAS (SGFANELGPRLMGK; SEQ ID NO: 11), T7 tag (MASMTGGQQMG; SEQ ID NO: 12), VSV-G tag (YTDIEMNRLGK; SEQ ID NO: 13), E-Tag (GAPVPYPDPLEPR; SEQ ID NO: 14), S-Tag (KETAAAKFERQHMDS; SEQ ID NO: 15), HSV tag (SQPELAPEDPED; SEQ ID NO: 16), KT3 tag (KPPTPPPEPET; SEQ ID NO: 17), TK15 tag, GST tag, Protein A tag, CD tag, Strep-Tag (WSHPQFEK; SEQ ID NO: 18), MBP tag, CBD tag, Avi tag (CGLNDIFEAQKIEWHE; SEQ ID NO: 19), CBP tag, TAP tag, and SF-TAP tag.

24. The method of claim 15, wherein the virus like particle (VLP) producing protein is a retroviral gag protein or a viral gag-like protein, optionally wherein the viral gag protein is selected from the group consisting of a murine leukemia virus (MLV) gag protein, a retrovirus matrix protein, a rhabdovirus matrix (M) protein (optionally VSVM protein), a filovirus viral core protein (optionally an Ebola VP40 viral protein), a Rift Valley Fever virus N protein (optionally RVFV N Protein having GenBank serial number NP049344), a coronavirus M, E and/or NP protein (optionally GenBank serial number NP040838 for NP protein, GenBank serial number NP 040835 for M protein, GenBank serial number CAC39303 for E protein of Avian Infections Bronchitis Virus and GenBank serial number NP828854 for E protein of the SARS virus), a bunyavirus N protein (optionally the bunyavirus N protein of GenBank serial number AAA47114), an influenza M1 protein, a paramyxovirus M protein, an arenavirus Z protein (optionally a Lassa Fever Virus Z protein), an AAV gag-like protein (optionally selected from the group consisting of AAV1 capsid, AAV2 capsid, AAV3 capsid, AAV4 capsid, AAV5 capsid, AAV6 capsid, AAV7 capsid, AAV8 capsid, AAV9 capsid, AAV10 capsid, AAV11 capsid, AAV12 capsid, and AAV13 capsid), and combinations thereof.

25. (canceled)

26. The method of claim 15, wherein:
the virus like particle (VLP) producing protein is a retroviral gag protein or a viral gag-like protein, optionally wherein the viral gag protein is selected from the group consisting of a murine leukemia virus (MLV) gag protein, a retrovirus matrix protein, a rhabdovirus matrix (M) protein (optionally VSVM protein), a filovirus viral core protein (optionally an Ebola VP40 viral protein), a Rift Valley Fever virus N protein (optionally RVFV N Protein having GenBank serial number NP049344), a coronavirus M, E and/or NP protein (optionally GenBank serial number NP040838 for NP protein, GenBank serial number NP 040835 for M protein, GenBank serial number CAC39303 for E protein of Avian Infections Bronchitis Virus and GenBank serial number NP828854 for E protein of the SARS virus), a bunyavirus N protein (optionally the bunyavirus N protein of GenBank serial number AAA47114), an influenza M1 protein, a paramyxovirus M protein, an arenavirus Z protein (optionally a Lassa Fever Virus Z protein), an AAV gag-like protein (optionally selected from the group consisting of AAV1 capsid, AAV2 capsid, AAV3 capsid, AAV4 capsid, AAV5 capsid, AAV6 capsid, AAV7 capsid, AAV8 capsid, AAV9 capsid, AAV10 capsid, AAV11 capsid, AAV12 capsid, and AAV13 capsid), and combinations thereof;
the epitope-tagged viral surface protein is a Vesicular Stomatitis Virus (VSV) glycoprotein (VSV-G) or a mutagenized form of VSV-G, optionally wherein the mutagenized form of VSV-G prevents VSV-G-mediated cellular uptake;
the epitope-tagged viral surface protein is an epitope-tagged viral envelope protein, optionally wherein the epitope-tagged viral envelope protein is selected from the group consisting of an epitope-tagged form of any of the following: a Vesicular Stomatitis Virus (VSV) glycoprotein, a retrovirus glycoprotein (optionally a human immunodeficiency virus (HIV) envelope glycoprotein (optionally HIVSF162 envelope glycoprotein of GenBank serial number M65024)), a simian immunodeficiency virus (SIV) envelope glycoprotein (optionally SIVmac239 envelope glycoprotein of GenBank serial number M33262), a simian-human immunodeficiency virus (SHIV) envelope glycoprotein (optionally SHIV-89.6p envelope glycoprotein of GenBank serial number U89134), a feline immunodeficiency virus (FIV) envelope glycoprotein (optionally FIV envelope glycoprotein of GenBank serial number L00607), a feline leukemia virus (FLV) envelope glycoprotein (optionally the FLV envelope glycoprotein of GenBank serial number M12500), a bovine immunodeficiency virus (BIV) envelope glycoprotein (optionally the BIV envelope glycoprotein of GenBank serial number NC001413), a bovine leukemia virus (BLV) envelope glycoprotein (optionally of GenBank serial number AF399703), an equine infectious anemia virus envelope glycoprotein (optionally the equine infectious anemia virus envelope glycoprotein of GenBank serial number NC001450), a human T-cell leukemia virus envelope glycoprotein (optionally the human T-cell leukemia virus envelope glycoprotein of GenBank serial number AF0033817), a mouse mammary tumor virus envelope glycoprotein (MMTV), a bunyavirus glycoprotein (optionally a Rift Valley Fever virus (RVFV) glycoprotein (optionally the RVFV envelope glycoprotein of GenBank serial number M11157)), an arenavirus glycoprotein (optionally a Lassa fever virus glycoprotein (optionally of GenBank serial number AF333969))), a filovirus glycoprotein (e.g., an Ebola virus glycoprotein (GenBank serial number NC002549)), a corona virus glycoprotein (optionally of GenBank serial number SARS coronavirus spike protein AAP13567), an influenza virus glycoprotein (optionally of GenBank serial number V01085), a paramyxovirus glycoprotein (optionally of GenBank serial number NC002728 for Nipah virus F and G proteins), a rhabdovirus glycoprotein (optionally of GenBank serial number NP049548)), an alphavirus glycoprotein (optionally of GenBank serial number AAA48370 for Venezuelan equine encephalomyelitis (VEE)), a flavivirus glycoprotein (optionally of GenBank serial number NC001563 for West Nile virus and/or a Hepatitis C Virus glycoprotein), a Herpes Virus glycoprotein (optionally a cytomegalovirus glycoprotein), and combinations thereof;
the epitope-tagged viral surface protein is selected from the group consisting of Coronavirus gpE1, Coronavirus Peplomer Protein E1, Coronavirus Peplomer Protein E2 JHM, Hepatitis Virus (MHV), Glycoprotein E2, LaCrosse Virus Envelope Glycoprotein G1, Simian Sarcoma Virus Glycoprotein 70, Viral Envelope Glycoprotein gp55 (Friend Virus), and Viral Envelope Glycoprotein gPr90 (Murine Leukemia Virus);
the epitope tag of the epitope-tagged viral surface protein is selected from the group consisting of FLAG (DYKDDDDK; SEQ ID NO: 3), 6×His (HHHHHH; SEQ ID NO: 4), HA (YPYDVPDYA; SEQ ID NO: 5), c-myc (EQKLISEEDL; SEQ ID NO: 6), V5 tag (GKPIPNPLLGLDST; SEQ ID NO: 7), AU1 tag (DTYRYI; SEQ ID NO: 8), AU5 tag (TDFYLK; SEQ ID NO: 9), Glu-Glu tag (EYMPME; SEQ ID NO: 10), OLLAS (SGFANELGPRLMGK; SEQ ID NO: 11), T7 tag (MASMTGGQQMG; SEQ ID NO: 12), VSV-G tag (YTDIEMNRLGK; SEQ ID NO: 13), E-Tag (GAPVPYPDPLEPR; SEQ ID NO: 14), S-Tag (KETAAAKFERQHMDS; SEQ ID NO: 15), HSV tag (SQPELAPEDPED; SEQ ID NO: 16), KT3 tag (KPPTPPPEPET; SEQ ID NO: 17), TK15 tag, GST tag, Protein A tag, CD tag, Strep-Tag (WSHPQFEK; SEQ ID NO: 18), MBP tag, CBD tag, Avi tag (CGLNDIFEAQKIEWHE; SEQ ID NO: 19), CBP tag, TAP tag, and SF-TAP tag;
the living cell is infected by a virus, optionally wherein the living cell is infected by AAV (and optionally adenovirus, HPV or other virus), or by a retrovirus, optionally wherein the retrovirus is a lentivirus;
the living cell is a mammalian cell, optionally a mammalian cell in culture;
the living cell is a neuronal cell, optionally a primary cortical neuron, optionally an excitatory neuron or an inhibitory neuron;
the living cell is a cell in vivo, optionally a living cell in a mouse model of disease, optionally a living cell in an engineered patient-derived xenograft (PDX) model for glioblastoma multiforme (GBM);
the living cell is a living cell in a rat, optionally a primary rat cortical neurons or a primary rat hippocampal neuron, optionally obtained from microsurgically dissected tissue, optionally from a E18 Sprague Dawley rat;
the first nucleic acid sequence and the second nucleic acid sequence are present on the same nucleic acid construct;
the first nucleic acid sequence and the second nucleic acid sequence are present on different nucleic acid constructs;
the first nucleic acid sequence and/or the second nucleic acid sequence are genomically integrated; and/or
the VLP producing protein and/or the epitope-tagged viral surface protein is under the control of a mammalian promoter, optionally a CMV promoter, a SV40 promoter and/or a tissue-specific mammalian promoter (optionally a mDIx, CamKII, Syn1, NSE, PDGF and/or Ta1 promoter, optionally a CamKII promoter and/or a mDIx promoter).

27-41. (canceled)

42. The method of claim 15, wherein:
the first VLP protein is specific to the first population of cells and the second VLP protein is specific to the second population of cells, optionally wherein the first VLP protein is the VLP producing protein encoded by the first nucleic acid and/or the second VLP protein is the VLP producing protein encoded by the second nucleic acid;
the binding of isolating step (e) is performed using an antibody and/or the binding of isolating step (g) is performed using an antibody;
the first VLP protein and/or the second VLP protein is tagged, optionally epitope-tagged; and/or
the analyte profile comprises transcript information.

43-47. (canceled)


SEQUENCE LISTING (28 sequences)

SEQ ID NO: 1 - protein, 538 aa, Artificial
  Synthetic
MGQAVTTPLSLTLDHWKDVERTAHNLSVEVRKRRWVTFCSAEWPTFNVGWPRDGTFNPDIITQVKIKVFSPGPHGHPDQVPYIVTWEAIAVDPPPWVRPFVHPKPPLSLPPSAPSLPPEPPLSTPPQSSLYPALTSPLNTKPRPQVLPDSGGPLIDLLTEDPPPYRDPGPPSPDGNGDSGEVAPTEGAPDPSPMVSRLRGRKEPPVADSTTSQAFPLRLGGNGQYQYWPFSSSDLYNWKNNNPSFSEDPAKLTALIESVLLTHQPTWDDCQQLLGTLLTGEEKQRVLLEARKAVRGEDGRPTQLPNDINDAFPLERPDWDYNTQRGRNHLVHYRQLLLAGLQNAGRSPTNLAKVKGITQGPNESPSAFLERLKEAYRRYTPYDPEDPGQETNVAMSFIWQSAPDIGRKLERLEDLKSKTLGDLVREAEKIFNKRETPEEREERIRRETEEKEERRRAEDVQREKERDRRRHREMSKLLATVVSGQRQDRQGGERRRPQLDHDQCAYCKEKGHWARDCPKKPRGPRGPRPQASLLTLDD

SEQ ID NO: 2 - protein, 511 aa, Artificial
  Synthetic
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 3 - protein, 8 aa, Artificial
  Synthetic
DYKDDDDK

SEQ ID NO: 4 - protein, 6 aa, Artificial
  Synthetic
HHHHHH

SEQ ID NO: 5 - protein, 9 aa, Artificial
  Synthetic
YPYDVPDYA

SEQ ID NO: 6 - protein, 10 aa, Artificial
  Synthetic
EQKLISEEDL

SEQ ID NO: 7 - protein, 14 aa, Artificial
  Synthetic
GKPIPNPLLGLDST

SEQ ID NO: 8 - protein, 6 aa, Artificial
  Synthetic
DTYRYI

SEQ ID NO: 9 - protein, 6 aa, Artificial
  Synthetic
TDFYLK

SEQ ID NO: 10 - protein, 6 aa, Artificial
  Synthetic
EYMPME

SEQ ID NO: 11 - protein, 14 aa, Artificial
  Synthetic
SGFANELGPRLMGK

SEQ ID NO: 12 - protein, 11 aa, Artificial
  Synthetic
MASMTGGQQMG

SEQ ID NO: 13 - protein, 11 aa, Artificial
  Synthetic
YTDIEMNRLGK

SEQ ID NO: 14 - protein, 13 aa, Artificial
  Synthetic
GAPVPYPDPLEPR

SEQ ID NO: 15 - protein, 15 aa, Artificial
  Synthetic
KETAAAKFERQHMDS

SEQ ID NO: 16 - protein, 12 aa, Artificial
  Synthetic
SQPELAPEDPED

SEQ ID NO: 17 - protein, 11 aa, Artificial
  Synthetic
KPPTPPPEPET

SEQ ID NO: 18 - protein, 8 aa, Artificial
  Synthetic
WSHPQFEK

SEQ ID NO: 19 - protein, 16 aa, Artificial
  Synthetic
CGLNDIFEAQKIEWHE

SEQ ID NO: 20 - DNA, 576 bp, Artificial
  Synthetic
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 21 - protein, 495 aa, Artificial
  Synthetic
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 22 - protein, 495 aa, Artificial
  Synthetic
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 23 - protein, 495 aa, Artificial
  Synthetic
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 24 - DNA, 3604 bp, Artificial
  Synthetic
acattgattattgactagttattaatagtaatcaattacggggtcattagttcatagcccatattggagttccgcgttacataacttacggtaaatggcccgcctggctgaccgcccaacgacccccgccccttgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttgtggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcgccaccatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgtcctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccgcagggatccggcgcaacaaacttctctctgctgaaacaagccggagatgtcgaagagaatcctggaccgatggagagcgacgagagcggcctgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagaccccggatgcagatgccggtgaagaa

SEQ ID NO: 25 - DNA, 3613 bp, Artificial
  Synthetic
acattgattattgactagttattaatagtaatcaattacggggtcattagttcatagcccatattggagttccgcgttacataacttacggtaaatggcccgcctggctgaccgcccaacgacccccgccccttgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttgtggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcgccaccatgaagtgccttttgtacttagcctttttattcattggggtgaattgcaagttcaccatagtttttccatccggaggagattacaaggatgacgacgataagggcggaagcttgggacacaaccaaaaaggaaactggaaaaatgttccttctaattaccattattgcccgtcaagctcagatttaaattggcataatgacttaataggcacagccttacaagtcaaaatgcccgcgagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaagcggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaaggaattcggatccggcgcaacaaacttctctctgctgaaacaagccggagatgtcgaagagaatcctggaccgatggtgtccaagggcgaggaagataacatggccatcatcaaggagttcatgaggtttaaggtccacatggagggttcagtcaatggccacgagttcgagattgaaggcgagggcgagggccgcccctacgaagggacacagacggcgaaattgaaggtgaccaaaggcgggccattgcccttcgcatgggacatcttgtcccctcagtttatgtatggcagcaaggcctatgttaagcaccccgctgatatcccggactacttgaagctgtcctttccagaggggtttaaatgggagcgcgttatgaatttcgaagacggaggagtggttacggtgacgcaggactcatccctgcaggacggagaatttatatataaggttaagttgagaggcacaaacttcccaagcgacggccctgtgatgcagaagaaaacaatggggtgggaagcttccagcgagcgcatgtaccccgaagatggcgccctcaagggcgagataaagcaaaggctgaaacttaaggacggcggtcattacgacgcggaggtcaagacaacttacaaggctaaaaaacccgttcagttgcctggggcttacaatgttaatatcaaacttgacatcacaagccacaatgaagactatacgatcgtggagcagtatgaacgagcggaaggcaggcactcaacgggggggatggacgagctttacaag

SEQ ID NO: 26 - DNA, 3616 bp, Artificial
  Synthetic
acattgattattgactagttattaatagtaatcaattacggggtcattagttcatagcccatattggagttccgcgttacataacttacggtaaatggcccgcctggctgaccgcccaacgacccccgccccttgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttgtggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcgccaccatgaagtgccttttgtacttagcctttttattcattggggtgaattgcaagttcaccatagtttttccatccggaggatacccatacgatgttccagattacgctggcggaagcttgggacacaaccaaaaaggaaactggaaaaatgttccttctaattaccattattgcccgtcaagctcagatttaaattggcataatgacttaataggcacagccttacaagtcaaaatgcccgcgagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaagcggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaaggaattcggatccggcgcaacaaacttctctctgctgaaacaagccggagatgtcgaagagaatcctggaccgatggtgtccaagggcgaggaagataacatggccatcatcaaggagttcatgaggtttaaggtccacatggagggttcagtcaatggccacgagttcgagattgaaggcgagggcgagggccgcccctacgaagggacacagacggcgaaattgaaggtgaccaaaggcgggccattgcccttcgcatgggacatcttgtcccctcagtttatgtatggcagcaaggcctatgttaagcaccccgctgatatcccggactacttgaagctgtcctttccagaggggtttaaatgggagcgcgttatgaatttcgaagacggaggagtggttacggtgacgcaggactcatccctgcaggacggagaatttatatataaggttaagttgagaggcacaaacttcccaagcgacggccctgtgatgcagaagaaaacaatggggtgggaagcttccagcgagcgcatgtaccccgaagatggcgccctcaagggcgagataaagcaaaggctgaaacttaaggacggcggtcattacgacgcggaggtcaagacaacttacaaggctaaaaaacccgttcagttgcctggggcttacaatgttaatatcaaacttgacatcacaagccacaatgaagactatacgatcgtggagcagtatgaacgagcggaaggcaggcactcaacgggggggatggacgagctttacaag

SEQ ID NO: 27 - DNA, 1237 bp, Artificial
  Synthetic
acattgattattgactagttattaatagtaatcaattacggggtcattagttcatagcccatattggagttccgcgttacataacttacggtaaatggcccgcctggctgaccgcccaacgacccccgccccttgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttgtggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggc

SEQ ID NO: 28 - protein, 495 aa, Artificial
  Synthetic
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK